(19) United States
(12) Patent Application Publication
(10) Pub. No.: US 2022/0202703 A1
(43) Pub. Date: Jun. 30, 2022

(54) IN SITU GEL-FORMING DELIVERY SYSTEMS, METHODS AND COMPOSITIONS

(71) Applicant: Poly-Med, Inc., Anderson, SC (US)

(72) Inventors: David Roper Ingram, Anderson, SC (US); Michael Scott Taylor, Anderson, SC (US); Kara Bethany Acampora, West Union, SC (US); Michael Aaron Vaughn, Anderson, SC (US); David Gravett, Mountain View, CA (US)

(21) Appl. No.: 17/439,348

(22) PCT Filed: Mar. 12, 2020
(86) PCT No.: PCT/US20/22241

§ 371 Date: Sep. 14, 2021

Related U.S. Application Data
(60) Provisional application No. 62/871,831, filed on Jul. 9, 2019.
(60) Provisional application No. 62/818,767, filed on Mar. 15, 2019.

(51) Int. Cl. A61K 9/00; A61K 31/498; A61K 47/34; A61K 47/44; A61K 47/10

(52) U.S. Cl. CPC A61K 9/0024; A61K 31/498; A61K 47/10; A61K 47/44; A61K 47/34

(57) ABSTRACT

In situ gel-forming compositions are disclosed, which may comprise one or more absorbable polymers, solvents such as N-methyl-2-pyrrolidone, polyethylene glycol or DMSO, and one or more bioactive agents. The composition forms a hydrogel or semi-solid mass on contact with an aqueous environment. Methods of using in situ gel-forming composition for various applications are also disclosed

### CROSS-REFERENCE TO RELATED APPLICATIONS

This application claims the benefit under 35 U.S.C. § 119(e) of U.S. Provisional Patent Application No. 62/818,767 filed Mar. 15, 2019, and U.S. Provisional Patent Application No. 62/871,831, filed Jul. 9, 2019, which applications are incorporated herein by reference in their entireties for all purposes.

### FIELD OF THE DISCLOSURE

The present disclosure relates generally to polymeric compositions for delivery of biologically active agents, and the manufacture and use thereof

### BACKGROUND

Reproducible sustained delivery of a drug at a target site is a goal of controlled drug-delivery systems. The most commonly used drug-delivery systems, which can release drugs longer than one week, are parenteral injections and implants. Certain implant systems can deliver drugs for more than one year, and the longest drug delivery can be achieved by biodegradable or nonbiodegradable implant systems.

Long-acting injectable formulations offer many advantages when compared with conventional formulations of the same compounds. These advantages include the following: a predictable drug-release profile during a defined period of time following each injection; better patient compliance; ease of application; improved systemic availability by avoidance of first-pass metabolism; reduced dosing frequency (i.e., fewer injections) without compromising the effectiveness of the treatment; decreased incidence of side effects; and overall cost reduction of medical care

Commonly used depot formulations are comprised of biodegradable polymer-excipients that control the rate of drug release and resorbs during/after drug release. A major advantage of depot delivery of active agents over oral medication is facilitation of compliance in medication taking. Biodegradable polymers have gained wide acceptance. The greatest advantage of these degradable polymers is that they are broken down into biologically acceptable molecules that are metabolized and removed from the body via normal metabolic pathways. This versatile delivery system offers the advantage of a very high loading and controlled release of various drug for an extended period of time compared with plain delivery system.

What is needed are new compositions of controlled, sustained release depot delivery of bioactive agents that offer advantages over older compositions in terms of convenience, side effect profiles, efficacy, and/or a fast onset of action.

### SUMMARY

In an aspect, the present disclosure provides an in situ gel-forming composition. The in situ gel-forming composition comprises an absorbable polymer, at least one bioactive agent, and a biocompatible solvent. The solvent dissolves the polymer and optionally, the agent, to create a fluid composition which may be administered to a subject in need thereof. The composition forms a hydrogel or semi-solid mass upon contact with an aqueous environment, such as occurs when the composition is administered to a subject in need of the bioactive agent, and releases the bioactive agent in a controlled manner. In additional aspects, the present disclosure provides methods of forming the composition and methods of using the composition for various applications, as well as kits containing the composition, as disclosed herein.

In an aspect, the present disclosure provides a composition, which is optionally a single phase composition, comprising an absorbable polymer, a solvent, and at least one bioactive agent such as a pharmaceutical or medicinal compound, nucleic acids, and/or a protein, optionally comprising two or more bioactive agents, where the composition has a viscosity of less than 15,000 cps to about 140,000 cps at room temperature and is biocompatible, where the composition forms a semi-solid mass upon administration to a subject in need thereof and releases the at least one bioactive agent(s) in a controlled manner. The semi-solid mass may also be described as a stable compliant depot which forms after injection of a composition of the present disclosure into a hydrated environment such as subcutaneous tissue. Optionally, one or more of the following exemplary features may be used to further characterize the composition, where other features are disclosed elsewhere herein: the protein is a component of a vaccine composition, the protein is an antigen, an antibody, or a fragment of an antibody, and/or the protein comprises two different proteins. A disclosed composition may further comprise a non-protein bioactive agent. An absorbable polymer may comprise two or more different absorbable polymers, and one or more absorbable polymer(s) may be a poly(ether-ester), poly(ether-carbonate) or poly(ether-ester-carbonate); an absorbable polymer may comprise a molecular chain having a X—Y—X or (X—Y)ₙ structure, wherein X represents a relatively hydrophobic polyester block, Y represents a relatively hydrophilic block, and n represents a number of repeating X—Y units. An X—Y—X or (X—Y)ₙ may be formed by grafting hydrophobic X blocks prepared from monomers selected from the group consisting of glycolide, lactide, E-caprolactone, p-dioxanone and trimethylene carbonate, to hydrophilic Y blocks selected from the group consisting of polyoxyethylene, poly(oxyethylene-b-oxypropylene), polypeptide polyalkylene oxamate, polysaccharide, derivatives thereof, and liquid, high molecular weight polyether glycols interlinked with an oxalate or a succinate functionality in linear or branched form. An absorbable polymer may comprise a segmented aliphatic polyurethane, for example, an absorbable polymer may comprise a segmented aliphatic polyurethane prepared from lactide and glycolide; or an absorbable polymer may comprise a segmented aliphatic polyurethane comprising polyoxyalkylene glycol chains covalently linked to polyester or polyester-carbonate chain segments, interlinked with aliphatic urethane segments. An absorbable polymer may comprise a molecular chain having a ([X—Y—X]—Z—)ₙ structure, wherein X represents a relatively hydrophobic polyester block, Y represents a relatively hydrophilic block, Z represents an aliphatic urethane segment and n represents a number of repeating entities. A solvent may comprise two non-identical solvents. A solvent includes, but is not limited to, dimethylsulfoxide (DMSO), ethyl lactate, ethyl acetate, benzyl alcohol, benzyl benzoate, triacetin, N-methylpyrrolidone (NMP), 2-pyrrolidone, propylene carbonate, polyethylene glycol (PEG200), polyethylene glycol (PEG400), dimethylformamide (DMF), or glycofurol. A disclosed composition may further comprise an oil, including but not limited to, corn oil, peanut oil, super-refined sesame oil, or super-refined peanut oil. A disclosed composition may further comprise a mono- or poly-hydric compound including, but not limited to, glycerol, 1,2-propanediol, or ethanol.

In an aspect, the present disclosure provides a method of controlled drug delivery comprising administering a composition disclosed herein to a subject in need thereof. For example, one bioactive agent in the composition may be a protein, for example, wherein the protein is optionally part of a vaccine. The composition administered according to the method may further comprise a second bioactive agent as described herein. Administering may comprise known routes of administration, including, but not limited to, intradermal, subcutaneous injection, intra-joint administration, intranasal, submucosal, subbuccal, topical, or other sites within or on a subject for which controlled drug delivery is desired.

The above-mentioned and additional features of the present disclosure and the manner of obtaining them will become apparent, and the disclosure will be best understood by reference to the following detailed description. All references disclosed herein are hereby incorporated by reference in their entirety as if each was incorporated individually.

This Brief Summary has been provided to introduce certain concepts in a simplified form that are further described in detail below in the Detailed Description. Except where otherwise expressly stated, this Brief Summary is not intended to identify key or essential features of the claimed subject matter, nor is it intended to limit the scope of the claimed subject matter.

The details of one or more aspects are set forth in the description below. The features illustrated or described in connection with one exemplary embodiment may be combined with the features of other aspects. Thus, any of the various aspects described herein can be combined to provide further aspects. Aspects of the aspects can be modified, if necessary to employ concepts of the various patents, applications and publications as identified herein to provide yet further aspects. Other features, objects and advantages will be apparent from the description, the drawings, and the claims.

### BRIEF DESCRIPTION OF THE DRAWINGS

Exemplary features of the present disclosure, its nature and various advantages will be apparent from the accompanying drawings and the following detailed description of various aspects. One or more aspects are described hereinafter with reference to the accompanying drawings in which:

FIG. 1 is a graph showing the effect of sterilization on the molecular weight of polymers incorporated into compositions of the present disclosure (n=3).

FIG. 2 is a graph showing the mass loss degradation profiles for compositions of the present disclosure, as measured over 70 days (n=4).

FIG. 3 is a graph showing the release of a hydrophilic drug (vancomycin hydrochloride) from compositions of the present disclosure, as measured over 25 days (n=3).

FIGS. 4A, 4B, and 4C are graphs showing primarily Newtonian rheological response of all compositions from 1-100 1/sec with shear thinning region from 100-1000 1/sec.

FIG. 5 is a graph showing release profiles up to 600 hours (˜25 days) for respective 6519L and 7017 compositions.

FIGS. 6A, 6B, and 6C are graphs of release of at least one bioactive agents by exemplary compositions disclosed herein. FIG. 6A is a graph showing mg bioactive agent/g composition/day of an exemplary composition disclosed herein. FIG. 6B is a graph showing mg bioactive agent/mL release buffer/day of an exemplary composition disclosed herein. FIG. 6C is a graph showing mg bioactive agent/day of an exemplary composition disclosed herein.

FIGS. 7, 8, 9 and 10 show release rate values for compositions disclosed herein comprising erdafitanib.

### DETAILED DESCRIPTION OF THE DISCLOSURE

The present disclosure may be understood more readily by reference to the following detailed description of preferred embodiments of the disclosure. In general, and unless defined otherwise, all technical and scientific terms used herein have the same meaning as commonly understood to one of ordinary skill in the art to which the presently disclosed subject matter belongs. However, certain terms have specific meanings as provided herein. In case of conflict, the present specification, including definitions, will control.

The singular forms “a,” “an,” and “the” are intended to include plural reference unless the context clearly dictates otherwise. For example, a solvent refers to both a single solvent as well as a mixture of two or more solvents. Likewise, a bioactive agent refers to both a single bioactive agent as well as a mixture of two or more bioactive agents. Similarly, unless the context clearly dictates otherwise, the letter “s” following a noun designates both the plural and singular forms of that noun. For example, polymers refer to a single polymer as well as a mixture of two or more polymers. The term “X and/or Y” means “X” or “Y” or both “X” and “Y”.

Where features or aspects of the disclosure are described in terms of a group of possible options, such as may be presented in a Markush group, it is intended, and those skilled in the art will recognize, that the disclosure embraces and is also thereby described in terms of any individual member and any subgroup of members of the group. Similarly, if a group of options is specified in order to describe a feature, that feature includes combinations of any two or more of the named options. For example, if a feature may be met by selecting A, B and C, that feature is met by selecting A alone, B alone, C alone, A and B in combination, A and C in combination, B and C in combination, and A, B and C in combination. Applicant reserves the right to revise the application or claims to refer specifically to any individual member or any subgroup or any combination of members of the group.

The term “effective amount” denotes the amount of a composition disclosed herein, such as a medicament or of a pharmaceutical active ingredient, which causes a biological or medical response in a tissue, system, animal or human which is sought or desired, for example, by a researcher or physician. In addition, the term “effective amount” denotes an amount which, compared with a corresponding subject who has not taken this amount, has the following consequence: modulated or improved treatment, healing, prevention or elimination of a disease, condition, syndrome, disease state, complaint, disorder or prevention of side effects or also the modulation or reduction in the progress of a disease, complaint or disorder. The term “effective amount” also encompasses the amounts which are effective for increasing normal physiological function.

The term “hydrogel” or “hydrogel mass” as used herein, refers to materials which have a high tendency for water absorption and/or retention, and maintain mechanical integrity through physical crosslinks which may be reversible in nature.

The term “polymer” has a conventional meaning and refers to macromolecules having one or more structurally unique repeating units, where the repeating units are arranged in a chain-like structure. A polymer formed from one unique repeating structural unit may be referred to as a homopolymer. A polymer formed entirely from, e.g., glycolide is an exemplary homopolymer. A polymer formed from two or more unique repeating structural units may be referred to as a copolymer. A polymer formed entirely from, e.g., glycolide and lactide is a copolymer. A tripolymer is a specific copolymer having three unique repeating structural units and a tetrapolymer is a specific copolymer having four unique repeating structural units. A copolymer may be linear or branched, and may be random or block. The term “polymer” as used herein refers to homopolymers, copolymers, tripolymers, tetrapolymers and other polymeric molecules made from monomeric subunits.

The term “semi-solid”, such as may be used to describe a material or composition, describes a material which in some respects has the properties of a solid, but in other respects has the properties of a liquid. A semi-solid material is similar to a solid in that it can support its own weight and hold its shape under the force of gravity. A semi-solid material is similar to a liquid in that it will flow under pressure to conform to the shape of the surface of the structure which is applying the pressure.

Additional definitions and conventions are provided herein.

In an aspect, the present disclosure provides a drug delivery system that is designed to address current challenges requiring controlled bioactive agent delivery. For example, a drug delivery system includes compositions comprising polymers that are formulated to allow localized controlled delivery which can be administered through known injectable or topical delivery devices. The drug delivery systems and compositions disclosed herein can be tailored to provide desired properties including one or more of viscosity, injectability, degradation profile, system sterilization and release of a drug, e.g., a hydrophilic drug.

### Absorbable Polymers

As used herein, the term “absorbable polymer” refers to a polymer that can be broken down by either chemical or physical process, upon interaction with a physiological environment, for example, at the implantation site within an animal subject, and that erodes, breaks down or dissolves within a period of time, e.g., within days, weeks or months. An absorbable polymer serves a temporary function in a subject, such as delivering a bioactive agent, for example, a drug. In situ, an absorbable polymer may degrade into fragments and be metabolized or excreted from the host. The term “absorbable polymer” may be used interchangeably herein with the terms “biodegradable polymer” and “bioabsorbable polymer”.

In an aspect, an absorbable polymer is a polyester, which refers to a polymer wherein all or substantially all repeating units are joined together by ester groups. Polyesters may be formed by reacting monomers having carboxyl and hydroxyl groups so as to form ester groups. Polyesters may be formed by ring opening polymerization of cyclic ester monomers. Polyesters may be formed from a monomer such as glycolide, lactide, ε-caprolactone, or p-dioxanone.

In an aspect, an absorbable polymer is an absorbable polyester copolymer, tripolymers, tetrapolymer or mixtures thereof. Suitable absorbable polyester copolymers include, but are not limited to, lactide/glycolide copolymers, ε-caprolactone/glycolide copolymers, lactide/trimethylene carbonate copolymers, lactide/glycolide/caprolactone tripolymers, lactide/glycolide/trimethylene carbonate tripolymers, lactide/caprolactone/trimethylene carbonate tripolymers, glycolide/ca prolactone/tri methylene carbonate tripolymers, and lactide/glycolide/caprolactone/trimethylene carbonate tetrapolymers. In an aspect, a polyester copolymer comprises a lactide/glycolide copolymer with a lactide/glycolide mole ratio of 60-90/40-10.

In an aspect, an absorbable polymer is a polyetherester, also referred to as a poly(etherester), which refers to a polymer wherein all or substantially all repeating units are joined together by either ester or ether groups, and where both ether and ester groups are present as linking groups in the polymer. A polyether/polyester polymer, also referred to as a poly(ether)(ester) polymer is a type of polyetherester which has a block copolymer structure comprising one or more blocks of repeating units joined together by ether groups, and one or more blocks of repeating units joined together by ester groups. In an aspect, an absorbable polymer is a poly(ether-carbonate) which refers to a polymer wherein all or substantially all repeating units are joined together by either ether or carbonate groups, and where both ether and carbonate groups are present as linking groups in the polymer. In an aspect, an absorbable polymer is a poly(ether-ester-carbonate) which refers to a polymer wherein all or substantially all repeating units are joined together by either ether, ester or carbonate groups, and where both ether, ester and carbonate groups are present as linking groups in the polymer.

In an aspect, an absorbable polymer comprises a molecular chain having a hydrophilic block, designated “Y” herein, and a relatively hydrophobic polyester block, designated “X” herein. The hydrophilic block, in isolated form, will be more soluble in water than is the hydrophobic block in isolated form. Optionally, an isolated form of the hydrophilic block is completely soluble in water at room temperature.

In an aspect, an hydrophobic block X comprises the polymerization product from a cyclic lactone, a cyclic carbonate polyalkylene oxalate, or the like. A cyclic lactone may include, but is not limited to, glycolide, l-lactide, dl-lactide, ε-caprolactone and p-dioxanone. A cyclic carbonate may include, but is not limited to, trimethylene carbonate. In an aspect, a hydrophobic block X is a polyester, for example, a polyester formed by reacting monomer including, but not limited to, glycolide, lactide, ε-caprolactone and p-dioxanone with an initiating group including, but not limited to, a hydroxyl or amino group. In an aspect, an hydrophobic block X is a poly(ester-carbonate), for example, a poly(ester-carbonate) formed by reacting monomer including, but not limited to, glycolide, lactide, ε-caprolactone, p-dioxanone together with another monomer, trimethylene carbonate, with an initiating group including, but not limited to, a hydroxyl or amino group.

In an aspect, a hydrophobic block is an absorbable polyester chain block(s) or segment(s) of variable length which, if present in an isolated form, will produce practically amorphous (with less than 5% crystallinity) or totally amorphous material having a T_{g} of less than 25° C., and optionally, is a viscous liquid at room temperature. For example, an hydrophobic segment or block X comprises lactide/glycolide copolymer made from 51 to 80 mol % I- or dl-lactide. In an aspect, an hydrophobic block is an absorbable polyester chain block(s) or segment(s) of variable length which, if present in an isolated form, will produce practically amorphous (with less than 5% crystallinity) or totally amorphous material having a T_{g} of less than 70° C.

In an aspect, a hydrophilic block, if present in an isolated form, may be water soluble. In an aspect, a hydrophilic block Y is a polyether. For example, a hydrophilic block or segment Y is or comprises poly(oxyethylene) or poly(oxyethylene-b-oxypropylene). The Y block may be formed from a liquid, high molecular weight polyether glycol interlinked with an oxalate or succinate functionalities in linear or branched form. Such hydrophilic segments or blocks may comprise a minor component of a higher homolog, such as, poly(oxypropylene)-polypeptide, e.g., polyalkylene oxamate. For example, a hydrophilic block Y is or comprises a polysaccharide including derivatives thereof. The length of the hydrophilic block and its weight fractions within the absorbable polymer can be varied to modulate the rate of gel formation, its modulus, its water content, diffusivity of bioactive drug through it, its adhesiveness to surrounding tissue, and bioabsorbability.

In an aspect, a hydrophobic block X and hydrophilic block Y comprise a molecular structure described by the formula X—Y—X. In an aspect, a hydrophobic block X and hydrophilic block Y comprise a molecular structure described by the formula or (X—Y)n where n denotes a number of repeating (X—Y) units. These X and Y blocks may be arranged in a linear or branched manner in the polymer.

In an aspect, an absorbable polymer comprises carboxylic end-groups formed by any known technique in the art, such as, for example, end-group succinylation and end-group acetylation. This facilitates ionically binding a biologically active agent or drug to the absorbable polymer such that the drug release can be modulated.

In an aspect, an absorbable polymer comprises a segmented, aliphatic polyurethane comprising polyoxyalkylene glycol chains covalently linked to polyester or polyester-carbonate chain segments, interlinked with aliphatic urethane segments. A polyoxyalkylene glycol chain may comprise at least one type of oxyalkylene sequences including, but not limited to, oxyethylene, oxypropylene, oxytrimethylene, or oxytetramethylene repeat units. In an aspect, a polyoxyalkylene glycol chain has an average molecular weight of 200-1200 Daltons. In an aspect, a polyoxyalkylene glycol chain is PEG 200, PEG 300, PEG 400, PEG 500, PEG 600, PEG 700, PEG 800, PEG 900, PEG 1000, where the number refers to the average molecular weight of the molecule in Daltons.

A polyester or polyester-carbonate chain segment may be derived from a cyclic monomer including, but not limited to, ε-caprolactone, trimethylene carbonate, p-dioxanone, 1,5-dioxepan-2-one, 1-lactide, dl-lactide, glycolide and morpholine dione. An aliphatic urethane segment may be derived from at least one diisocyanate including, but not limited to, hexamethylene diisocyanate, lysine-derived diisocyanate, and cyclohexane bis(methylene isocyanate).

In an aspect, a segmented, aliphatic polyurethane has an ether/ester mass ratio of 20-49/80-51, preferably 25-40/75-55 and, most preferably 30-40/70-60. In an aspect, a segmented, aliphatic polyurethane is prepared by forming a prepolymer and then reacting the prepolymer with monomer to form the final polymer. In such a case, the final polymer may have a prepolymer/diisocyante mass ratio in the range of 1:0.5 to 1:1.4. In an aspect, a segmented, aliphatic polyurethane has a prepolymer/diisocyante molar ratio of 1:0.66, 1:0.8 or 1:1.2 as measured by the initiator/diisocyanate molar ratio.

In an aspect, an absorbable polymer is a segmented polyether-carbonate-urethane (PECU), which has one or more of the following characteristics: (a) exhibits <20 percent, or <15 percent, or <10 percent, or <5 percent, or no solubility in water; (b) is a liquid at about 50° C.; (c) has a weight average molecular weight exceeding 10 kDa; (d) swells in an aqueous environment leading to an increase of volume of at least 3 percent, and (e) is miscible in biocompatible organic solvents such as N-methylpyrrolidone (NMP), polyethylene glycol or dimethylsulfoxide (DMSO), to facilitate use as an injectable composition that undergoes gel-formation when introduced to aqueous biological sites. A segmented polyether-carbonate-urethane (PECU) is relatively slow-absorbing in situ compared to PLGA, 10:90, lactide: glycolide.

In an aspect, an absorbable polymer is a segmented aliphatic polyether-ester urethane (PEEU). In an aspect, a PEEU has one or more of the following characteristics: (a) exhibits <20 percent, or <15 percent, or <10 percent, or <5 percent, or no solubility in water; (b) is a liquid at about 50° C.; (c) has a weight average molecular weight exceeding 10 kDa; (d) swells in an aqueous environment leading to an increase of volume of at least 3 percent, and (e) is miscible in biocompatible organic solvents such as NMP, polyethylene glycol or DMSO, to facilitate use as an injectable composition that undergo gel-formation when introduced to aqueous biological sites. A segmented aliphatic polyether-ester urethane (PEEU) exhibits relatively fast absorption in situ compared to PLGA 10:90 (lactide:glycolide).

In an aspect, an absorbable polymer is a segmented aliphatic polyether-carbonate-ester urethane (PECEU). In an aspect, a PECEU possess one or more of the following features: (a) exhibits <20 percent, or <15 percent, or <10 percent, or <5 percent, or no solubility in water; (b) is a liquid at about 50° C.; (c) has a weight average molecular weight exceeding 10 kDa; (d) swells in an aqueous environment leading to an increase of volume of at least 3 percent, and (e) is miscible in biocompatible organic solvents such as NMP, polyethylene glycol or DMSO, to facilitate their use as injectable compositions that undergo gel-formation when introduced to aqueous biological sites. A segmented aliphatic polyether-carbonate-ester urethane (PECEU) exhibits relatively fast absorption in situ compared to PEEU.

In an aspect, an absorbable polymer is a segmented, aliphatic polyether-ester urethanes (APEEU) and polyether-ester-carbonate urethanes (APEECU). Typical APEEUs and APEECUs comprise polyoxyalkylene chains (such as those derived from polyethylene glycol and block or random copolymers of ethylene oxide and propylene oxide) covalently linked to polyester or polyester-carbonate segments (derived from at least one monomer including, but not limited to, trimethylene carbonate, ε-caprolactone, lactide, glycolide, p-dioxanone, 1,5-dioxepan-2-one, and a morpholinedione) and interlinked with aliphatic urethane segments derived from 1,6 hexamethylene-, 1-4 cyclohexane-, cyclohexane-bis-methylene-, 1,8 octamethylene- or lysine-derived diisocyanate.

### Bioactive Agent

Compositions of the present disclosure include at least one pharmaceutical or medicinal compound or molecule, referred to herein as a bioactive agent. The biologically active agent or bioactive agent may also be referred to as an active pharmaceutical ingredient (API) or a drug. As noted previously, a biologically active agent refers to one, as well as more than one biologically active agent. A bioactive agent may be described in terms of its biological function or its chemical class. An exemplary bioactive agent includes, but is not limited to, a/an antiandrogen, antibacterial, antioestrogen, androgen or anabolic agent, antibiotic, antimigraine drug, antihistamine, antianxiety drug, antidiuretic, antihistamine, antirheumatoid agent, antigen, analgesic, antidepressant, antiinflammatory, anesthetic, aminoglycoside, antibody, antibody fragment, antiviral, adrenergic stimulant, anticonvulsant, antiangina agent, antiarrhyrthmic, antimalarial, anti-mitotic agent, anthelmintic, anoretic agent, antitussive, antipruritic, antipyretic, anti-Alzheimer's agent, anti-Parkinson's agent, antiemetic and antinauseant, antihypertensive, anticoagulant, antifungal, antimicrobial, allergen, antidiarrheal, antihyperuricaemia agent, adrenergic stimulant, antiparasitic agent, antiproliferative agent, antipsychotic drug, antithyroid agent, beta-adrenergic blocking agent, bronchodilator, bronchospasm relaxant, blood clotting factor, blood coagulation factor, cytotoxic agent, cytostatic agent, chemotherapeutic, clot inhibitor, clot dissolving agent, cell, CNS stimulant, corticosteroid, calcium channel blocker, cofactor, ceramide, cardiotonic glycoside, cytokine (e.g., lymphokine, monokine, chemokine), colony stimulating factor (e.g., GCSF, GM-CSF, MCSF), dermatological agent, decongestant, diuretic, expectorant, endectocide agent, growth factor, growth factor receptor, growth factor receptor inhibitor, hemostatic agent, hypoglycemic agent, hormone or hormone analog, hypercalcemia, hypnotic, interleukin (IL-2, IL-3, IL-4, IL-6); interferon (e.g., β-IFN, α-IFN and γ-IFN), immunosuppressant, muscle relaxant, microorganism, non-steroidal anti-inflammatory agent, nucleic acid, nutritional agent, neuromuscular blocking agent, neuroleptic, neurotoxin, nutraceutical, oligonucleotide, oestrogen, obstetric drug, ovulation inducer, opioid, opioid agonist or antagonist progestogen, pituitary hormone, pituitary inhibitor protein, peptide, polysaccharide, protease inhibitor, prostaglandin, quinolone, reductase inhibitor, sulfa drug, sclerosant, sedative, sodium channel blockers, steroid, steroidal anti-inflammatory agent, smoking cessation agent, toxin, thrombolytic agent, thyroid hormone, tumor necrosis factor; vesicle, vitamin, mineral, virus, vasodilator, or a vaccine. Exemplary bioactive agents include the following options.

A bioactive agent may comprise antidiarrheals such as diphenoxylate, loperamide and hyoscyamine. A bioactive agent may comprise antihypertensives such as hydralazine, minoxidil, captopril, enalapril, clonidine, prazosin, debrisoquine, diazoxide, guanethidine, methyldopa, reserpine, trimethaphan. A bioactive agent may comprise calcium channel blockers such as diltiazem, felodipine, amlodipine, nitrendipine, nifedipine and verapamil. A bioactive agent may comprise antiarrhyrthmics such as amiodarone, flecainide, disopyramide, procainamide, mexiletene and quinidine. A bioactive agent may comprise antiangina agents such as glyceryl trinitrate, erythrityl tetranitrate, pentaerythritol tetranitrate, mannitol hexanitrate, perhexilene, isosorbide dinitrate and nicorandil. A bioactive agent may comprise beta-adrenergic blocking agents such as alprenolol, atenolol, bupranolol, carteolol, labetalol, metoprolol, nadolol, nadoxolol, oxprenolol, pindolol, propranolol, sotalol, timolol and timolol maleate.

A bioactive agent may comprise cardiotonic glycosides such as digoxin and other cardiac glycosides and theophylline derivatives. A bioactive agent may comprise adrenergic stimulants such as adrenaline, ephedrine, fenoterol, isoprenaline, orciprenaline, rimeterol, salbutamol, salmeterol, terbutaline, dobutamine, phenylephrine, phenylpropanolamine, pseudoephedrine and dopamine. A bioactive agent may comprise vasodilators such as cyclandelate, isoxsuprine, papaverine, dipyrimadole, isosorbide dinitrate, phentolamine, nicotinyl alcohol, co-dergocrine, nicotinic acid, glycerl trinitrate, pentaerythritol tetranitrate and xanthinol. A bioactive agent may comprise antiproliferative agents such as paclitaxel, estradiol, actinomycin D, sirolimus, tacrolimus, everolimus, 5-fluorouracil, Gemcitabine and dexamethasone.

A bioactive agent may comprise antimigraine preparations such as ergotanmine, dihydroergotamine, methysergide, pizotifen and sumatriptan. A bioactive agent may comprise anticoagulants and thrombolytic agents such as warfarin, dicoumarol, low molecular weight heparins such as enoxaparin, streptokinase and its active derivatives. A bioactive agent may comprise hemostatic agents such as aprotinin, tranexamic acid and protamine.

A bioactive agent may comprise analgesics and antipyretics including the opioid analgesics such as buprenorphine, dextromoramide, dextropropoxyphene, fentanyl, alfentanil, sufentanil, hydromorphone, methadone, morphine, oxycodone, papaveretum, pentazocine, pethidine, phenopefidine, codeine, dihydrocodeine; acetylsalicylic acid (aspirin), paracetamol, synthetic alpha2-adrenoreceptor agonist, dexmedetomidine hydrochloride, flunixin meglumine, meperidine, phenylbutazone and phenazone. A bioactive agent may include an agonist or antagonist of a known opioid compound.

A bioactive agent may comprise immunosuppressants, antiproliferatives and cytostatic agents such as rapamycin (sirolimus) and its analogs (everolimus and tacrolimus). A bioactive agent may comprise neurotoxins such as capsaicin and botulinum toxin (botox). A bioactive agent may comprise hypnotics and sedatives such as the barbiturates amylobarbitone, butobarbitone and pentobarbitone and other hypnotics and sedatives such as chloral hydrate, chlormethiazole, hydroxyzine and meprobamate. A bioactive agent may not comprise disulfram. A bioactive agent may comprise disulfram. A bioactive agent may comprise antianxiety agents such as the benzodiazepines alprazolam, bromazepam, chlordiazepoxide, clobazam, chlorazepate, diazepam, flunitrazepam, flurazepam, lorazepam, nitrazepam, oxazepam, temazepam and triazolam. A bioactive agent may comprise compounds effective in treating addiction, including but not limited to, acamprosate, topiramate, naltrexone, or nalmefene. A bioactive agent may comprise BSA (bovine serum albumin).

A bioactive agent may comprise neuroleptic and antipsychotic drugs such as the phenothiazines, chlorpromazine, fluphenazine, pericyazine, perphenazine, promazine, thiopropazate, thioridazine, trifluoperazine; and butyrophenone, droperidol and haloperidol; and other antipsychotic drugs such as pimozide, thiothixene and lithium. A bioactive agent may comprise antidepressants such as the tricyclic antidepressants amitryptyline, clomipramine, desipramine, dothiepin, doxepin, imipramine, nortriptyline, opipramol, protriptyline and trimipramine and the tetracyclic antidepressants such as mianserin and the monoamine oxidase inhibitors such as isocarboxazid, phenelizine, tranylcypromine and moclobemide and selective serotonin re-uptake inhibitors such as fluoxetine, paroxetine, citalopram, fluvoxamine and sertraline. A bioactive agent may comprise central nervous system (CNS) stimulants such as caffeine and 3-(2-aminobutyl) indole.

A bioactive agent may comprise antipruritics such as synthetic Janus Kinase (JAK) inhibitors, NK-1 receptor antagonists, antibodies that neutralize interleukin-31 (IL-31). These can include oclacitinib maleate, Serlopitant and Lokivetmab. A bioactive agent may comprise anti-Alzheimer's agents such as tacrine. A bioactive agent may comprise anti-Parkinson's agents such as amantadine, benserazide, carbidopa, levodopa, benztropine, biperiden, benzhexol, procyclidine and dopamine-2 agonists such as S (−)-2-(N-propyl-N-2-thienylethylamino)-5-hydroxytetralin (N-0923). A bioactive agent may comprise anticonvulsants such as phenytoin, valproic acid, primidone, phenobarbitone, methylphenobarbitone and carbamazepine, ethosuximide, methsuximide, phensuximide, sulthiame and clonazepam.

A bioactive agent may comprise antiemetics and antinauseants such as the phenothiazines prochloperazine, thiethylperazine, a neurokinin (NK1) receptor antagonist, maropitant citrate and 5HT-3 receptor antagonists such as ondansetron and granisetron, as well as dimenhydrinate, diphenhydramine, metoclopramide, domperidone, hyoscine, hyoscine hydrobromide, hyoscine hydrochloride, clebopride and brompride. A bioactive agent may comprise non-steroidal anti-inflammatory agents including their racemic mixtures or individual enantiomers where applicable, preferably which can be formulated in combination with dermal and/or mucosal penetration enhancers, such as ibuprofen, flurbiprofen, ketoprofen, aclofenac, diclofenac, aloxiprin, aproxen, aspirin, diflunisal, fenoprofen, indomethacin, mefenamic acid, naproxen, phenylbutazone, piroxicam, salicylamide, salicylic acid, sulindac, desoxysulindac, tenoxicam, tramadol, ketoralac, flufenisal, salsalate, triethanolamine salicylate, aminopyrine, antipyrine, oxyphenbutazone, apazone, cintazone, flufenamic acid, clonixerl, clonixin, meclofenamic acid, 6-chloro-α-methyl-9H-carbazole-2-acetic acid (carprofen), flunixin, coichicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, and triflumidate.

A bioactive agent may comprise antirheumatoid agents such as penicillamine, aurothioglucose, sodium aurothiomalate, methotrexate and auranofin. A bioactive agent may comprise muscle relaxants such as baclofen, diazepam, cyclobenzaprine hydrochloride, dantrolene, methocarbamol, orphenadrine and quinine. A bioactive agent may comprise agents used to treat gout and hyperuricaemia such as allopurinol, colchicine, probenecid and sulphinpyrazone. A bioactive agent may comprise oestrogens such as estradiol, estriol, estrone, ethinylestradiol, mestranol, stilbestrol, dienestrol, epiestriol, estropipate and zeranol.

A bioactive agent may comprise progesterone and other progestagens such as allylestrenol, dydrgesterone, lynestrenol, norgestrel, norethyndrel, norethisterone, norethisterone acetate, gestodene, levonorgestrel, medroxyprogesterone and megestrol. A bioactive agent may comprise antiandrogens such as cyproterone acetate and danazol. A bioactive agent may comprise antioestrogens such as tamoxifen and epitiostanol and the aromatase inhibitors, exemestane and 4-hydroxy-androstenedione and its derivatives. A bioactive agent may comprise androgens and anabolic agents such as testosterone, methyltestosterone, clostebol acetate, drostanolone, furazabol, nandrolone oxandrolone, stanozolol, trenbolone acetate, dihydro-testosterone, 17-(α-methyl-19-noriestosterone and fluoxymesterone.

A bioactive agent may comprise 5-α Reductase inhibitors such as finasteride, turosteride, LY-191704 and MK-306. A bioactive agent may comprise corticosteroids such as betamethasone, betamethasone valerate, cortisone, dexamethasone, dexamethasone 21-phosphate, fludrocortisone, flumethasone, fluocinonide, fluocinonide desonide, fluocinolone, fluocinolone acetonide, fluocortolone, halcinonide, halopredone, hydrocortisone, hydrocortisone 17-valerate, hydrocortisone 17-butyrate, hydrocortisone 21-acetate, methylprednisolone, prednisolone, prednisolone 21-phosphate, prednisone, triamcinolone and triamcinolone acetonide.

A bioactive agent may comprise glycosylated proteins, proteoglycans, and glycosaminoglycans such as chondroitin sulfate; chitin, acetyl-glucosamine and hyaluronic acid. A bioactive agent may comprise complex carbohydrates such as glucans.

A bioactive agent may comprise steroidal anti-inflammatory agents such as cortodoxone, fludroracetonide, fludrocortisone, difluorsone diacetate, flurandrenolone acetonide, medrysone, amcinafel, amcinafide, betamethasone and its other esters, chloroprednisone, clorcortelone, descinolone, desonide, dichlorisone, difluprednate, flucloronide, flumethasone, flunisolide, flucortolone, fluoromethalone, fluperolone, fluprednisolone, meprednisone, methylmeprednisolone, paramethasone, cortisone acetate, hydrocortisone cyclopentylpropionate, cortodoxone, flucetonide, fludrocortisone acetate, flurandrenolone, aincinafal, amcinafide, betamethasone, betamethasone benzoate, chloroprednisone acetate, clocortolone acetate, descinolone acetonide, desoximetasone, dichlorisone acetate, difluprednate, flucloronide, flumethasone pivalate, flunisolide acetate, fluperolone acetate, fluprednisolone valerate, paramethasone acetate, prednisolamate, prednival, triamcinolone hexacetonide, cortivazol, formocortal and nivazol.

A bioactive agent may comprise pituitary hormones and their active derivatives or analogs such as corticotrophin, thyrotropin, follicle stimulating hormone (FSH), a gonadotropin-releasing hormone (GnRH) analog, deslorelin acetate, cetrorelix acetate, gonadorelin acetate, clomiphene, human chorionic gonadotropin (HCG), luteinizing hormone (LH) and gonadotrophin releasing hormone (GnRH).

A bioactive agent may comprise hypoglycemic agents such as insulin, chlorpropamide, glibenclamide, gliclazide, glipizide, tolazamide, tolbutamide and metformin. A bioactive agent may comprise thyroid hormones such as calcitonin, thyroxine and liothyronine and antithyroid agents such as carbimazole and propylthiouracil. A bioactive agent may comprise hormone agents such as octreotide. A bioactive agent may comprise pituitary inhibitors such as bromocriptine. A bioactive agent may comprise ovulation inducers such as clomiphene.

A bioactive agent may comprise diuretics such as the thiazides, related diuretics and loop diuretics, bendrofluazide, chlorothiazide, chlorthalidone, dopamine, cyclopenthiazide, hydrochlorothiazide, indapamide, mefruside, methycholthiazide, metolazone, quinethazone, bumetanide, ethacrynic acid and frusemide and potassium sparing diuretics, spironolactone, amiloride and triamterene. A bioactive agent may comprise antidiuretics such as desmopressin, lypressin and vasopressin including their active derivatives or analogs. A bioactive agent may comprise obstetric drugs including agents acting on the uterus such as ergometrine, oxytocin and gemeprost. A bioactive agent may comprise prostaglandins such as alprostadil (PGE1), prostacyclin (PGI2), dinoprost (prostaglandin F2-alpha) and misoprostol.

A bioactive agent may comprise antimicrobials including the cephalosporins such as cephalexin, cefoxytin and cephalothin. A bioactive agent may comprise penicillins such as amoxycillin, amoxycillin with clavulanic acid, ampicillin, bacampicillin, benzathine penicillin, benzylpenicillin, carbenicillin, cloxacillin, methicillin, phenethicillin, phenoxymethylpenicillin, flucloxacillin, meziocillin, piperacillin, ticarcillin and azlocillin. A bioactive agent may comprise tetracyclines such as minocycline, chlortetracycline, tetracycline, demeclocycline, doxycycline, methacycline and oxytetracycline and other tetracycline-type antibiotics. A bioactive agent may comprise amnioglycoides such as amikacin, amikin sulfate, gentamicin, kanamycin, neomycin, netilmicin and tobramycin. A bioactive agent may comprise rifampin, or antimicrobial peptide (AMP), specifically the synthetic peptide hLF (1-11).

A bioactive agent may comprise antifungals such as amorolfine, isoconazole, clotrimazole, econazole, miconazole, nystatin, terbinafine, bifonazole, amphotericin, griseofulvin, ketoconazole, fluconazole and flucytosine, salicylic acid, fezatione, ticlatone, tolnaftate, triacetin, zinc, pyrithione and sodium pyrithione. A bioactive agent may comprise quinolones such as nalidixic acid, cinoxacin, ciprofloxacin, enoxacin and norfloxacin; Sulphonamides such as phthalysulphthiazole, sulfadoxine, sulphadiazine, sulphamethizole and sulphamethoxazole. A bioactive agent may comprise sulphones such as dapsone.

A bioactive agent may comprise antibiotics such as chloramphenicol, clindamycin, erythromycin, erythromycin ethyl carbonate, erythromycin estolate, erythromycin glucepate, erythromycin ethylsuccinate, erythromycin lactobionate, roxithromycin, lincomycin, natamycin, nitrofurantoin, spectinomycin, vancomycin, aztreonarn, colistin IV, metronidazole, tinidazole, fusidic acid, trimethoprim, and 2-thiopyridine N-oxide; halogen compounds, particularly iodine and iodine compounds such as iodine-PVP complex and diiodohydroxyquin, hexachlorophene; chlorhexidine; chloroamine compounds; Lincomycin Hydrochloride, tricyclic tetrahydroquinoline antibacterial agents, 8-pyrazinyl-S-spiropyrimidinetrione-oxazinoquinoline derivatives, 3-spiropyrimidinetrione-quinoline derivatives, thiadiazol-spiropyrimidinetrione-quinoline derivatives, (2R,4S,4aS)-10-fluoro-2,4-dimethyl-8-(4-methyloxazol-2-yl)-2,4,4a,6-tetrahydro-1H,1′H-spiro[[1,4]oxazino[4,3-a]quinoline-5,5′-pyrimidine]-2′,4′,6′(-3′H)-trione, (2R,4S,4aS)-9,10-difluoro-2,4-dimethyl-8-(3-methylisoxazol-5-yl)-2,4,4a,6-tetrahydro-1H,1′H-spiro[[1,4]oxazino[4,3-a]quinoline-5,5′-pyrimidine]-2′,-4′,6′(3′H)-trione, (2R,4S,4aS)-10-fluoro-2,4-di methyl-8-(oxazol-2-yl)-2,4,4a,6-tetrahydro-1H-,1′H-spiro[[1,4]oxazino[4,3-a]quinoline-5,5′-pyrimidine]-2′,4′,6′(3′H)-tri-one, (2R,4S,4aS)-9,10-difluoro-2,4-dimethyl-8-(2-methyloxazol-5-yl)-2,4,4a,64-etrahydro-1H,1′H-spiro[[1,4]oxazino[4,3-a]quinoline-5,5′-pyrimidine]-2′,4′-,6′(3′H)-trione, (2R,4S,4aS)-9,10-difluoro-2,4-dimethyl-8-(oxazol-4-yl)-2,4,4a,6-tetra hydr-o-1H,1′H-spiro[[1,4]oxazino[4,3-a]quinoline-5,5′-pyrimidine]-2′,4′,6′(3′H)-trione, (2R,4S,4aS)-9-fluoro-2,4-dimethyl-8-(4-methyloxazol-2-yl)-2,4,4a,6-tetrah-ydro-1H,1′H-spiro[[1,4]oxazino[4,3-a]quinoline-5,5′-pyrimidine]-2′,4′,6′(3-′H)-trione, (2R,4S,4aS)-9,10-difluoro-8-(4-(4-fluorophenyl)oxazol-5-yl)-2,4-dimethyl-2,4,4a,6-tetrahydro-1H,1′H-spiro[[1,4]oxazino[4,3-a]quinoline-5,5′-pyrimidine]-2′,4′,6′(3′H)-trione, (2S,4R,4aR)-2,4-dimethyl-8-(oxazol-5-yl)-2,4,4a,6-tetrahydro-1H,1′H-spiro-[[1,4]oxazino[4,3-a]quinoline-5,5′-pyrimidine]-2′,4′,6′(3′H)-trione, (2S,4R,4aR)-8-(4-ethyloxazol-2-yl)-9,10-difluoro-2,4-dimethyl-2,4,4a,6-te-trahydro-1H,1′H-spiro[[1,4]oxazino[4,3-a]quinoline-5,5′-pyrimidine]-2′,4′,-6′(3′H)-trione, (2R,4S,4aS)-9,10-difluoro-2,4-dimethyl-8-(oxazol-2-yl)-2,4,4a,6-tetrahydr-o-1H,1′H-spiro[[1,4]oxazino[4,3-a]quinoline-5,5′-pyrimidine]-2′,4′,6′(3′H)-trione and benzoyl peroxide.

A bioactive agent may comprise antituberculosis drugs such as ethambutol, isoniazid, pyrazinamide, rifampicin and clofazimine. A bioactive agent may comprise antimalarials such as primaquine, pyrimethamine, chloroquine, hydroxychloroquine, quinine, mefloquine and halofantrine. A bioactive agent may comprise compounds including Azithromycin, Aztreonam, Cefaclor, Cefadroxil, Cefazolin, Cefdinir, Cefepime Hydrochloride, (cefoperazone sodium, Ceftaroline fosamil, avibactam, Ceftazidime sodium, Ceftibuten, ceftiofur, Tazobactam, cefovecin sodium[(6R,7R)-7-[[(2Z)-(2-amino-4-thiazolyl)(methoxyimino)acetyl]amino]-8-oxo-3-[(2S)-tetrahydro-2-furanyl]-5-thia-1-azabicyclo[4.2.0]oct-2-ene-2-carboxylic acid, monosodium salt] Cefuroxime Axetil, Cefuroxime, Cephalexin, Chloramphenicol Sodium, Ciprofloxacin HCl, Clarithromycin, Clindamycin hydrochloride, Clindamycin Palmitate hydrochloride, Clindamycin phosphate, Dalbavancin Hydrochloride, Daptomycin, Demeclocycline hydrochloride, Dicloxacillin, Doripenem, Doxycycline, Doxycycline calcium, Doxycycline hyclate, Doxycycline monohydrate, Ertapenem sodium, Erythromycin, Erythromycin Ethylsuccinate, Erythromycin lactobionate, Erythromycin stearate, Erythromycin, Fosfomycin tromethamine, Gemifloxacin mesylate, Gentamicin Sulfate, Imipenem, Kanamycin, Levofloxacin, Lincomycin hydrochloride, Linezolid, Meropenem, Methenamine Hippurate, Metronidazole, Metronidazole, Micafungin sodium, Minocycline Hydrochloride, Minocycline, Moxifloxacin hydrochloride, Nafcillin, Nalidixic acid, Neomycin Sulfate, Nitrofurantoin, Norfloxacin, Ofloxacin, Oritavancin diphosphate, Oxacillin, Penicillin G, Penicillin G benzathine, Penicillin G Sodium, Penicillin V Potassium, Piperacillin Sodium, Polymyxin B Sulfate, Quinupristin, dalfopristin, Spectinomycin hydrochloride, Streptomycin, Sulfamethoxazole, Tedizolid Phosphate, Telavancin, Telithromycin, Tetracycline Hydrochloride, Ticarcillin disodium, Tigecycline, Tobramycin Sulfate, Tobramycin, Trimethoprim hydrochloride, tulathromycin and Vancomycin hydrochloride.

A bioactive agent may comprise antiviral agents such as acyclovir and acyclovir prodrugs, famcyclovir, zidovudine, didanosine, stavudine, lamivudine, zalcitabine, saquinavir, indinavir, ritonavir, n-docosanol, tromantadine and idoxuridine. A bioactive agent may comprise anthelmintics such as mebendazole, thiabendazole, niclosamide, praziquantel, pyrantel embonate and diethylcarbamazine. A bioactive agent may comprise cytotoxic agents such as plicamycin, cyclophosphamide, dacarbazine, fluorouracil and its prodrugs (described, for example, in International Journal of Pharmaceutics, 111, 223-233 (1994)), methotrexate, procarbazine, Gemcitabine, 6-mercaptopurine and mucophenolic acid.

A bioactive agent may comprise anorectic and weight reducing agents including dexfenflurarnine, fenfluramine, diethylpropion, mazindol and phentermine. A bioactive agent may comprise agents used in treating hypercalcaemia such as calcitriol, dihydrotachysterol and their active derivatives or analogs. A bioactive agent may comprise antitussives such as ethylmorphine, dextromethorphan and pholcodine.

A bioactive agent may comprise antiparasitic and endectocide agents such as moxidectin, Ivermectin, Niclosamide, Praziquantel, Pyrantel, Pyrvinium, Albendazole, Flubendazole, Mebendazole and Thiabendazole. A bioactive agent may comprise expectorants such as carbolcysteine, bromihexine, emetine, quanifesin, ipecacuanha and saponins. A bioactive agent may comprise decongestants such as phenylephrine, phenylpropanolamine and pseudoephedrine.

A bioactive agent may comprise bronchospasm relaxants such as ephedrine, fenoterol, orciprenaline, rimiterol, salbutamol, sodium cromoglycate, cromoglycic acid and its prodrugs (described, for example, in International Journal of Pharmaceutics 7, 63-75 (1980)), terbutaline, ipratropium bromide, salmeterol and theophylline and theophylline derivatives.

A bioactive agent may comprise antihistamines such as meclozine, cyclizine, chlorcyclizine, hydroxyzine, brompheniramine, chlorpheniramine, clemastine, cyproheptadine, dexchlorpheniramine, diphenhydramine, diphenylamine, doxylamine, mebhydrolin, pheniramine, tripolidine, azatadine, diphenylpyraline, methdilazine, terfenadine, astemizole, loratidine and cetirizine.

A bioactive agent may comprise local anaesthetics such as benzocaine, bupivacaine, amethocaine, lignocaine, lidocaine, cocaine, cinchocaine, dibucaine, mepivacaine, prilocaine, etidocaine, veratridine (specific c-fiber blocker) and procaine. A bioactive agent may comprise stratum corneum lipids such as ceramides, cholesterol and free fatty acids, for improved skin barrier repair (Man, et al. J. Invest. Dermatol., 106(5), 1096, (1996)). A bioactive agent may comprise neuromuscular blocking agents such as suxamethonium, alcuronium, pancuronium, atracurium, gallamine, tubocurarine and vecuronium.

A bioactive agent may comprise sclerosing agents or sclerosants which may be a surfactant, or it may be selected from the group consisting of ethanol, dimethyl sulfoxide, sucrose, sodium chloride, dextrose, glycerin, minocycline, tetracycline, doxycycline, polidocanol, sodium tetradecyl sulfate, sodium morrhuate and sotradecol. A bioactive agent may comprise an angiogenesis inhibitor. A bioactive agent may comprise a 5-lipoxygenase inhibitor or antagonist. A bioactive agent may comprise a chemokine receptor antagonist.

A bioactive agent may comprise a cell cycle inhibitor such as a taxane; an anti-microtubule agent; paclitaxel; an analogue or derivative of paclitaxel; a vinca alkaloid; camptothecin or an analogue or derivative thereof; a podophyllotoxin, wherein the podophyllotoxin may be an etoposide or an analogue or derivative thereof; an anthracycline, wherein the anthracycline may be doxorubicin or an analogue or derivative thereof or the anthracycline may be mitoxantrone or an analogue or derivative thereof; a platinum compound; a nitrosourea; a nitroimidazole; a folic acid antagonist; a cytidine analogue; a pyrimidine analogue; a fluoropyrimidine analogue; a purine analogue; a nitrogen mustard or an analogue or derivative thereof; a hydroxyurea; a mytomicin or an analogue or derivative thereof, for example mitomycin A, mitomycin B, and mitomycin C; an alkyl sulfonate; a benzamide or an analogue or derivative thereof; a nicotinamide or an analogue or derivative thereof; a halogenated sugar or an analogue or derivative thereof; a DNA alkylating agent; an anti-microtubule agent; a topoisomerase inhibitor; a DNA cleaving agent; an antimetabolite; a nucleotide interconversion inhibitor; a hydroorotate dehydrogenase inhibitor; a DNA intercalation agent; an RNA synthesis inhibitor; a pyrimidine synthesis inhibitor; a cyclin dependent protein kinase inhibitor; an epidermal growth factor kinase inhibitor; an elastase inhibitor; a factor Xa inhibitor; a farnesyltransferase inhibitor; a fibrinogen antagonist; a guanylate cyclase stimulant; a heat shock protein 90 antagonist; which may be a geldanamycin or an analogue or derivative thereof; a guanylate cyclase stimulant; a HMGCoA reductase inhibitor, which may be simvastatin or an analogue or derivative thereof; an IKK2 inhibitor; an IL-1 antagonist; an ICE antagonist; an IRAK antagonist; an IL-4 agonist; an immunomodulatory agent; sirolimus or an analogue or derivative thereof; everolimus or an analogue or derivative thereof; tacrolimus or an analogue or derivative thereof; biolmus or an analogue or derivative thereof; tresperimus or an analogue or derivative thereof; auranofin or an analogue or derivative thereof; 27-0-demethylrapamycin or an analogue or derivative thereof; gusperimus or an analogue or derivative thereof; pimecrolimus or an analogue or derivative thereof; ABT-578 or an analogue or derivative thereof; an inosine monophosphate dehydrogenase (IMPDH) inhibitor, which may be mycophenolic acid or an analogue or derivative thereof or 1-α-25 dihydroxy vitamin D₃ or an analogue or derivative thereof; a leukotriene inhibitor; an MCP-1 antagonist; an MMP inhibitor; an NF kappa B inhibitor, which may be Bay 11-7082; an NO antagonist; a p38 MAP kinase inhibitor, which may be SB 202190; a phosphodiesterase inhibitor; a TGF-β inhibitor; a thromboxane A2 antagonist; a TNF-α antagonist; a TACE inhibitor; a tyrosine kinase inhibitor; vitronectin inhibitor; a fibroblast growth factor inhibitor; a protein kinase inhibitor; a PDGF receptor kinase inhibitor; an endothelial growth factor receptor kinase inhibitor; a retinoic acid receptor antagonist; a platelet derived growth factor receptor kinase inhibitor; a fibrinogen antagonist; an antimycotic agent; sulconizole; a bisphosphonate; a phospholipase A1 inhibitor; a histamine H1/H2/H3 receptor antagonist; a macrolide antibiotic; a GPIIb/IIIa receptor antagonist; an endothelin receptor antagonist; a peroxisome proliferator-activated receptor agonist; an estrogen receptor agent; a somastostatin analogue; a neurokinin 1 antagonist; a neurokinin 3 antagonist; a VLA-4 antagonist; an osteoclast inhibitor; a DNA topoisomerase ATP hydrolyzing inhibitor; an angiotensin I converting enzyme inhibitor; an angiotensin II antagonist; an enkephalinase inhibitor; a peroxisome proliferator-activated receptor gamma agonist insulin sensitizer; a protein kinase C inhibitor; a ROCK (rho-associated kinase) inhibitor; a CXCR3 inhibitor; Itk inhibitor; a cytosolic phospholipase A₂-α inhibitor; a PPAR agonist; an immunosuppressant; an Erb inhibitor; an apoptosis agonist; a lipocortin agonist; a VCAM-1 antagonist; a collagen antagonist; an α-2 integrin antagonist; a TNF-α inhibitor; a nitric oxide inhibitor; and a cathepsin inhibitor.

A bioactive agent may comprise anti-fibrin and fibrinolytic agents including plasmin, streptokinase, single chain urokinase, urokinase, t-PA (tissue type plasminogen activator) and aminocaproic acid. A bioactive agent may comprise anti-platelet agents including aspirin and prostacyclins (and analogues). A bioactive agent may comprise glycoprotein IIb/IIIa agents including monoclonal antibodies and peptides (e.g. ReoPro, Cilastagel, eptifibatide, tirofiban, ticlopidine, Vapiprost, dipyridamole, forskolin, angiopeptin, argatroban).

A bioactive agent may comprise thromboxane inhibitors; anti-thrombin and anti-coagulant agents, including dextan, heparin, LMW heparin (Enoxaparin, Dalteparin), hirudin, recombinant hirudin, anti-thrombin, synthetic antithrombins, thrombin inhibitors, Warfarin (and other coumarins).

A bioactive agent may comprise anti-mitotic, antiproliferative and cytostatic agents, including vincristine, vinblastine, paclitaxel, methotrexate, cisplatin, fluorouracil, Gemcitabine, rapamycin, azathioprine, cyclophosphamide, mycophenolic acid, corticosteroids, colchicine, nitroprusside; antiangiogenic and angiostatic agents, including paclitaxel, angiostatin and endostatin. A bioactive agent may comprise ACE inhibitors (e.g. Cilazapril, Lisinopril, Captopril).

A bioactive agent may comprise growth factor (e.g. VEGF, FGF) antagonists. A bioactive agent may comprise growth factor receptor inhibitor. A bioactive agent may comprise an FGF receptor (FGFR) inhibitor. Such bioactive agents may be useful for treating one or more types of cancer. For example, urothelial cancer, most frequently in the bladder, is the sixth most common type of cancer in the U.S. It is estimated that in 2018, 81,190 new cases of bladder cancer will be diagnosed in the U.S. and an estimated 17,240 bladder cancer deaths will occur. The relative five-year survival rate for patients with Stage IV metastatic bladder cancer is currently five percent. Patients with metastatic urothelial cancer, who have FGFR genetic alterations, have poor prognoses and a high unmet need based on low response rates and may be resistant to treatment with immune-checkpoint inhibitors. Erdafitinib, C₂₅H₃₀N₆O₂, which may also be known as 1,2-Ethanediamine, N1-(3,5-dimethoxyphenyl)-N2-(1-methylethyl)-N1-(3-(1-methyl-1H-pyrazol-4-yl)-6-quinoxalinyl)-is an investigational, once-daily oral pan-fibroblast growth factor receptor (FGFR) inhibitor being studied in Phase 2 and Phase 3 clinical trials for the treatment of patients with locally advanced or metastatic urothelial cancer. FGFRs are a family of receptor tyrosine kinases, which can be activated by genetic alterations in a variety of tumor types, and these alterations may lead to increased tumor cell growth and survival. FGFRs are a subset of tyrosine kinases which are unregulated in some tumors and influence tumor cell differentiation, proliferation, angiogenesis, and cell survival. Erdafitinib is being evaluated for safety and efficacy in phase II clinical trials for cholangiocarcinoma, gastric cancer, non-small cell lung cancer, and esophageal cancer.

Methods disclosed herein comprise administering an effective amount of a composition disclosed herein for treatment of urothelial cancer, bladder cancer, cholangiocarcinoma, gastric cancer, non-small cell lung cancer, and esophageal cancer. For example, a composition may comprise erdafitinib, C₂₅H₃₀1\1₆O₂, as a bioactive agent. In an aspect, a composition disclosed herein may comprise erdafitinib and a absorbable polymer 7017, disclosed herein. Compositions may comprise derivatives of erdafitinib, salts of erdfitinib, pharmaceutical diluents or excipients, and other known additives in formulations comprising erdafitinib. Other FGFR inhibitors that can be used include infigratinib (BGJ398) and AZD4547 (AstraZeneca). Other kinase inhibitors that can be used include imatinib, ponatinib, pazopanib and trametinib.

A bioactive agent may comprise antioxidants, minerals, and vitamins (e.g. Probucol, Tocopherol, Vitamins A, C, B1, B2, B6, B 12, B 12-alpha, and E, vitamin E acetate and vitamin E sorbate, calcium, magnesium, iron, copper, selenium); calcium channel blockers (e.g. nifedipine); fish oil (omega 3-fatty acid); phosphodiesterase inhibitors (e.g. dipyridamole); nitric acid donors (e.g. Molsidomine); somatostatin analogues (e.g., angiopeptin); immunosuppresives and anti-inflammatory agents (e.g. prednisolone, glucocorticoid and dexamethasone); radionuclides such as α, β and γ emitting isotopes (e.g. Re-188, Re-186, 1-125, Y-90); COX-2 inhibitors such as Celecoxib and Vioxx; kinase inhibitors such as epidermal growth factor kinase inhibitor, tyrosine kinase inhibitors, MAP kinase inhibitors protein transferase inhibitors, Resten-NG; smoking cessation agents such as nicotine, bupropion and ibogaine; insecticides and other pesticides which are suitable for local application; vitamins A, C, B1, B2, B6, B 12, B 12-alpha, and E, vitamin E acetate and vitamin E sorbate.

A bioactive agent may comprise allergens for desensitisation such as house, dust or mite allergens, grasses, trees, pollens, food molecules, sensitizing chemicals, and other known allergens; nutritional agents and nutraceuticals, such as vitamins, essential amino acids and fats; macromolecular pharmacologically active agents such as proteins, enzymes, peptides, polysaccharides (such as cellulose, amylose, dextran, chitin), nucleic acids, cells, tissues, and the like; bone mending biochemicals such as calcium carbonate, calcium phosphate, hydroxyapetite or bone morphogenic protein (BMP); angiogenic growth factors such as Vascular Endothelial Growth Factor (VEGF) and epidermal growth factor (EFG); cytokines interleukins; fibroblasts; cytotaxic chemicals; keratolytics such as the alpha-hydroxy acids, glycolic acid and salicylic acid; DNA, RNA or other oligonucleotides or polynucleotides.

A bioactive agent may comprise vaccines, including vaccines known and used for humans and animals. For example, human-related vaccines, including, but are not limited to, measles, mumps, varicella, polio, pertussis, typhoid, staphylococcus, and those vaccines for oncogenic treatments (e.g., poliovirus for glioblastoma) or genetic transformative vaccines, (e.g., AAV or adenovirus. For example, vaccines for animals include, but are not limited to, Hendra virus (HeV) G glycoprotein and/or Nipah virus G glycoprotein, Lutenising Hormone Releasing Hormone (LHRH) peptide, LHRH-diphtheria toxoid conjugate, porcine circovirus type 2 (PCV2) antigen, a porcine reproductive and respiratory syndrome virus antigen, Mycoplasma hyopneumoniae protein antigen, proteins or protein fragments, for example ORFI Torque teno virus protein, or other TTV proteins or fragments, antigens against Aeromonas salmonicida, antigens against Vibrio anguillarum, and antigens against V. salmonicida.

A bioactive agent may comprise growth factors such as Vascular Endothelial Growth Factor (VEGF) and epidermal growth factor (EFG), Fibroblast Growth Factors (FGF-1 through FGF-23), Interleukins (IL-1 through IL-13), Insulin-like Growth Factor-1, platelet derived growth factor (PDGF), nerve growth factors, neutrophins [Brain-derived neurotrophic factor (BDNF), Nerve growth factor (NGF), Neurotrophin-3 (NT-3), Neurotrophin-4 (NT-4)], Transforming growth factors (TGF-α, TGF-β), Tumor necrosis factor (TNF); and growth factor agonists or antagonists as well as antibodies against these growth factors. A bioactive agent may be an inhibitor of a receptor for the above growth factors.

In an aspect, a bioactive agent may be insoluble in a disclosed composition. For example, a bioactive agent may be present in the composition as a microparticulate dispersion. In an aspect, a bioactive agent may be deposited as a coating on the surface of an absorbable particle, e.g., a microparticle, where the particle or microparticle may be porous or microporous, and a disclosed composition further comprises the microparticle or particle. In an aspect, a bioactive agent may ionically bound onto the surface of an absorbable particle, e.g., a microparticle, where the particle or microparticle may be porous or microporous, and is incorporated into a disclosed composition.

In an aspect, a bioactive agent is a protein, where that term includes peptides and polypeptides, sugar-modified protein such as glycoprotein, as well as functional descriptions of protein classes such as antigen, enzyme, immunoglobulin and antibody. The composition may include a special delivery vehicle for the bioactive agent, such as a virus or modified virus, where the bioactive agent, such as a protein or polynucleotide, is contained within or expressed by the special delivery vehicle.

Where the protein has a net charge, for example a net positive charge, an absorbable polymer may have a complementary charge, for example an absorbable polymer may have a net negative charge and can bind a bioactive agent with a net positive charge. In this way, the bioactive agent will be attracted by ionic charge interaction to the absorbable polymer, and is thus slowly released from the in situ deposited composition. Alternatively, when faster release of a bioactive agent is desired, an absorbable polymer may have the same net charge as the bioactive agent. For instance, if the bioactive agent has a net negative charge, then the absorbable polymer will also have a net negative charge, and the bioactive agent will be quickly released from the in situ gelled composition.

Compositions of the present disclosure include at least one bioactive agent. The amount of bioactive agent relative to the amount of the polymer in the composition can be varied based on the specific activity of the bioactive agent and the intended duration of delivery. In an aspect, a bioactive agent-to-polymer (w/w) ratio is in the range of about 5:95 to 60:40, 10:90 to 50:50, 10:90 to 20:80, or 15:85 to 30:70, or 20:80 to 50:50 w/w.

### Solvent

Compositions of the present disclosure comprise a polymer, a bioactive agent and a solvent. The solvent is miscible with, e.g., it may be able to dissolve, the polymer. The combination of solvent and polymer is a fluid that can, for example, be injected into a subject in need thereof. In addition to being miscible with the polymer, the solvent is biocompatible. Compositions of the present disclosure, upon contact with an aqueous environment, change from a fluid state to a semi-solid mass.

Examples of solvents that can be used in compositions disclosed herein include, but are not limited to, dimethylsulfoxide, ethyl lactate, ethyl acetate, benzyl alcohol, benzyl benzoate, triacetin, N-methylpyrrolidone, 2-pyrrolidone, propylene carbonate, polyethylene glycol (PEG200), polyethylene glycol (PEG400), polyethylene glycols PEG 200-1000, dimethylformamide (DMF) and glycofurol.

In an aspect, a composition may further comprise an oil. Exemplary oils include corn oil, peanut oil, super refined sesame oil, and super refined peanut oil.

In an aspect, a composition may further comprise a mono- or poly-hydric compound. Exemplary mono- and poly-hydric compounds include glycerol, 1,2-propanediol and ethanol.

In an aspect, a composition may further comprise tributyl citrate, triethyl citrate, diethyl citrate, acetyl tributyl citrate, and acetyl triethyl citrate.

### Polymer-To-Solvent Ratio

The polymer-to-solvent ratio can be modulated to alter characteristics of particular drug delivery systems, for example, in concert with a bioactive agent's solubility, an intended release site, gelation rate and release rate. In an aspect, a polymer-to-solvent (w/w) ratio is in the range of about 5:95 to 55:45, 10:90 to 50:50, 10:90 to 20:80, or 15:85 to 30:70, or 20:80 to 50:50 w/w. In an aspect, a solvent comprises NMP and the polymer-to-solvent (w/w) ratio is in the range of 10:90 to 50:50, 10:90 to 20:80, or 15:85 to 30:70. In an aspect, a solvent comprises PEG and the polymer-to-solvent (w/w) ratio is in the range of 50:50 to 70:30. In an aspect, a solvent comprises methyl acetate and/or ethyl acetate, and the polymer-to-solvent (w/w) ratio is in the range of 10:90 to 50:50. In an aspect, a solvent comprises caprolactone monomer and the polymer-to-solvent (w/w) ratio is in the range of 40:60 to 60:40. In an aspect, a solvent comprises DMSO and the polymer-to-solvent (w/w) ratio is in the range of 70:30 to 90:10. In an aspect, a polymer can comprise two or more different solvents. In an aspect, a polymer-to-solvent 1 (w/w) ratio is in the range of about 5:95 to 55:45, 10:90 to 50:50, 10:90 to 20:80, or 15:85 to 30:70, or 20:80 to 50:50 w/w and the (polymer/solvent 1)-to-solvent 2 ratio is in the range of about 5:95 to 55:45, 10:90 to 50:50, 10:90 to 20:80, or 15:85 to 30:70, or 20:80 to 50:50 w/w.

### Solid Absorbable Carrier

In an aspect, an in situ gel-forming composition disclosed herein may further comprises a solid absorbable carrier to carry the one or more bioactive agent. The one or more bioactive agent/drug can be deposited, wholly or in part, on one or more types of solid absorbable carriers. In an aspect, a solid absorbable carrier is an absorbable, microporous low molecular weight polyester which is highly crystalline and practically insoluble in the absorbable polymer of the in situ gel-forming composition.

In an aspect, an in situ gel-forming composition disclosed herein comprises the solid carrier and the absorbable polymer at a weight ratio of 20/80, with the carrier being a low molecular, microporous polyglycolide with 0.70 to 0.95 solid fraction, average particle size of 0.5-200 micron and carboxyl-bearing chains. High concentration of carboxylic groups on the chains can be achieved by preparing the solid carrier using di- or poly-carboxylic acid as initiators. The deposited agent on the solid carrier can exhibit a release profile which can be multiphasic, including: (a) simple, fast diffusion of soluble free drug through the in situ hydrogel; (b) slow diffusion of soluble free drug housed in the pores of the solid carrier; and, (c) drug release at the surface (both exterior and pore) of the solid carrier or the chain ends of carboxylated chains by ion exchange of ionically bound molecules. By varying the concentration of the solid carrier in the in situ gel-forming composition, the flow characteristics and release profile of the bioactive agent can be modulated.

In an aspect, an absorbable carrier comprises microspheres or nanoparticles, such as biodegradable polylactic acid (PLA) microspheres, for controlled drug delivery. Other suitable biodegradable polymers include, but are not limited to, polyglycolic acid (PGA), lactic acid-glycolic acid copolymer (PLGA), poly-ε-caprolactone (PCL), lactic acid-ε-caprolactone copolymer (PLCL), polydioxanone (PDO), polytrimethylene carbonate (PTMC), poly(amino acid), polyanhydride, polyorthoester and copolymers thereof. The microspheres or nanoparticles can be prepared by polymerizing the monomeric mixture under known polymerization conditions in the presence of one or more bioactive agents such that the bioactive agent(s) is entrapped in the polymerized product.

### Viscosity

In certain embodiments, the in situ gel-forming composition is a liquid at room temperature that can be easily administered through known injection devices such as a syringe needle, a tube, a cannula, a trocar or a catheter. The treatment can be a single administration or multiple administrations that are separated by a period of time. A disclosed composition can be administered by known routes of administration, including, but not limited to, topical, subcutaneous, intra-dermal, intra-muscular, submucosal, sublingual, subbuccal, and other routes of administration.

In an aspect, compositions disclosed herein are sterile and pharmaceutically acceptable for injection into or application onto subjects, such as humans or animals. As used herein, subjects include living organisms, including humans, mammals, animals, fish, crustaceans, arthropods, plants and members of the known kingdoms Monera, Protists, Fungi, Animals and Plants, or domains. For example, a disclosed composition is contained in a prefilled syringe or vial in a sterile condition. In an aspect, a disclosed composition in a prefilled syringe or vial and is stable at 2-8° C. or 20-25° C. for at least 6 months, 12 months or 24 months.

In an aspect, an in situ gel-forming composition disclosed herein comprises an injectable liquid at room temperature. As used hereinafter, the term “injectable liquid” refers to a liquid that can be administered into a recipient through an injection device commonly used in the medical arts, such as needles, syringes and catheters. In an aspect, an injectable liquid composition has a viscosity that allows the liquid to be administered through a 10 gauge needle without excessive force. In an aspect, an injectable liquid has a viscosity that allows the liquid to be administered through a 23 gauge needle without excessive force. In an aspect, an injectable liquid composition has a viscosity that allows the liquid to be administered through a catheter of dimensions 2.4 FR, 2.5FR, 4.0FR, 4.8FR or 6.0FR. The viscosity for each liquid composition may be slightly different for each catheter size to facilitate administration of the product.

In an aspect, a composition of the present disclosure is an injectable liquid having a viscosity at room temperature of less than 500,000 cP. In an aspect, a composition of the present disclosure is an injectable liquid having a viscosity at room temperature of less than 250,000 cP. In an aspect, a composition of the present disclosure is an injectable liquid having a viscosity at room temperature of less than 100,000 cP. In an aspect, a composition of the present disclosure is an injectable liquid having a viscosity at room temperature of less than 50,000 cP. In an aspect, a composition of the present disclosure is an injectable liquid having a viscosity at room temperature of less than 25, 000 cP.

In an aspect, a composition of the present disclosure is an injectable liquid having a viscosity at room temperature of less than 15,000 cps, or less than 14,000 cps, or less than 13,000 cps, or less than 12,000 cps, or less than 11,000 cps, or less than 10,000 cps, or less than 9,000 cps, or less than 8,000 cps, or less than 7,000 cps, or less than 6,000 cps, or less than 5,000 cps, or less than 4,000 cps, or less than 3,000 cps, or less than 2,000, or less than 1,000 cps. The viscosity may be described as following within a range with any of the forgoing viscosity values as a maximum viscosity value and a minimum viscosity value of at least 100 cps, or at least 500 cps, or at least 1,000 cps, or at least 1,500 cps, or at least 2,000 cps, or at least 2,500 cps, or at least 3,000 cps.

In an aspect, a composition of the present disclosure is an injectable liquid that can be injected using a force of less than 300N, or less than 200N, or less than 100N, or less than 50N, or less than 25N, or less than 15N, or less than 10N.

### Rate of Gelation and Rate of Absorption

In an aspect, an in situ gel-forming composition of the present disclosure is formulated for rapid gelation at the treatment site. In an aspect, an in situ gel-forming composition is capable of forming a hydrogel or semi-solid mass on or at a treatment site in a period of 1-120 seconds, 1-5 minutes, 5-15 minutes, or 15-30 minutes. In an aspect, an in situ gel-forming composition disclosed herein forms a hydrogel or semi-solid mass on or at a treatment site within 15 seconds, 30 seconds, 60 seconds or 90 seconds. In an aspect, a gelation time is determined visually by observing the period between injection and formation of the solid mass. Upon exposure to an aqueous environment, the diluent/solvent portion of a composition may precipitate leaving the hardened polymeric hydrogel or semi-solid mass that can be visually detected or can be palpated or touched. In other cases, the diluent/solvent portion of a composition may diffuse away from the composition, leaving the hardened polymeric hydrogel or semi-solid mass that can be visually detected or can be palpated or touched.

The rate of gelation may be modulated by adding one or more gelation accelerators to an in situ gel-forming composition. Compounds that may serve as gelation accelerators include, but are not limited to, collagen, thrombin, activated platelets, chitosan, fibrinogen and antifibrinolytics.

In an aspect, an in situ gel-forming composition of the present disclosure is formulated for slow degradation at the treatment site. In an aspect, an in situ gel-forming composition of the present disclosure is formulated for a degradation time of 1-3 weeks, 1-3 months, 3-6 months, or 6-12 months through utilization of different polymer configurations.

### Methods of Preparation

The composition of the present disclosure can be prepared by several different methods. In an aspect, the polymer can be dissolved in a volatile solvent. The bioactive agent is then added to the polymer-solvent solution which is thoroughly mixed to disperse the bioactive agent throughout the polymer-solvent system. In an aspect the bioactive agent is soluble in the solvent and a solution is formed. In another aspect the bioactive agent may only be partially soluble in the solvent and thus a solution/dispersion is produced. In another aspect, the bioactive agent is poorly or not soluble in the solvent in which case the bioactive agent is dispersed in the polymer-solvent mixture. The volatile solvent can then be removed using heat, reduced pressure, gas flow or a combination thereof. The polymer-bioactive agent composition can be used without the addition of any additional solvents.

In an aspect, the bioactive agent is dissolved or dispersed in a volatile solvent. The bioactive agent-solvent can be added to the polymer. The polymer with then dissolve in the solvent to provide a polymer-solvent-bioactive agent composition. The volatile solvent can then be removed using heat, reduced pressure, gas flow or a combination thereof. In an aspect, the bioactive agent is dissolved or dispersed in a volatile solvent. The polymer is separately dissolved in a volatile solvent. The solvent used to dissolve or disperse the bioactive agent may or may not be the same solvent that is used to dissolve the polymer. Once the bioactive agent is uniformly distributed throughout the composition, the volatile solvent or solvents can be removed using heat, reduced pressure, gas flow or a combination thereof. Volatile solvents can include but are not limited to alcohols such as methanol, ethanol or isopropyl alcohol, ketones such as acetone or methyl ethyl ketone, ethers such as tetrahydrofuran, methyl tert-butyl ether or diethyl ether, aromatic solvents such as toluene xylene, or benzene, halogenated solvents such a dichloromethane, chloroform or hexafluoroisopropyl alcohol (HFIP), or solvents such as acetonitrile. One or more solvents can be used to incorporate the bioactive agent into the polymer.

In an aspect, the polymer may be dissolved in a non-volatile solvent. The bioactive agent can be added directly to the polymer-solvent composition. In an aspect, the polymer may be dissolved in a non-volatile solvent and the bioactive agent can be dissolved or dispersed separately in a volatile solvent. After combining the two mixtures, the volatile solvent can then be removed using heat, reduced pressure, gas flow or a combination thereof. In an aspect, the polymer can be dissolved in a non-volatile solvent, and the bioactive agent can be separately dissolved or dispersed in a non-volatile solvent. The two mixtures can then be combined. The non-volatile solvent used to dissolve the polymer, may be the same as the non-volatile solvent that is used to dissolve or disperse the bioactive agent or the solvent used for the polymer may be different to that used for the bioactive agent. More than one solvent may be used for the polymer as well as the bioactive agent. An exemplary method to prepare a composition of the present disclosure is provided. A pre-selected weighed amount of absorbable polymer is heated to 50° C. then mixed thoroughly at that temperature with a solvent to provide a homogeneous solution. The homogeneous solution is cooled to room temperature and then combined with a solution of bioactive agent and a solvent, which may or may not be the same solvent used to dissolve the absorbable polymer. If a volatile solvent is used to pre-dissolve the bioactive agent, then after the combination of the bioactive agent and absorbable polymer have been thoroughly mixed, the volatile solvent may be removed by applying reduced pressure to the composition.

The present disclosure comprises a kit comprising an in situ gel-forming composition of the present disclosure or its components, and instructions for using the in situ gel-forming composition. In an aspect, a kit comprises an in situ gel-forming composition packaged in a pre-filled syringe or vial. In an aspect, an in situ gel-forming composition contained in a prefilled syringe or vial is sterile. In an aspect, an in situ gel-forming composition contained in a prefilled syringe or vial is stable at 2-8° C. or 20-25° C. for at least 6 months, 12 months or 24 months. In an aspect the kit can comprise the polymer-solvent composition that is packaged separately from the bioactive agent. The bioactive agent and the polymer-solvent composition can be mixed prior to administration of the combined polymer-solvent-bioactive agent composition.

The polymer-bioactive agent compositions of the present disclosure can be incorporated into or onto a drug delivery platform or a medical device. The drug delivery platform can include but is not limited to a tube, foam, patch or sponge. A medical device can include but is not limited to a mesh, stent, catheter, suture or braid, and a pump (osmotic or mechanical).

### Methods of Use

An aspect of the present disclosure comprises methods for treating various pathologies, diseases and conditions using an in situ gel-forming composition of the present disclosure. A method comprises the steps of administering, e.g., by injection or application, to a subject, in need of such treatment, an effective amount of an in situ gel-forming composition as described herein comprising absorbable polymer, one or more solvents, and one or more bioactive agents, wherein said in situ gel-forming composition forms a hydrogel or semi-solid mass on contact with an aqueous environment at a treatment site. The in situ gel-forming composition can be injected or applied directly from a syringe or through a needle, trocar, catheter, tube, or cannula, or other dispensing elements.

For example, in an aspect the present disclosure provides a method for drug (bioactive agent) delivery. The method comprises the step of administering into a subject, an effective amount of an in situ gel-forming composition comprising an absorbable polymer, a solvent, and a drug, optionally in combination with biodegradable microspheres or nanoparticles comprising the drug, wherein said in situ gel-forming composition forms a hydrogel or semi-solid mass on contact with an aqueous environment on or at a treatment site.

As one illustrative example, the method comprises treatment of cancer or a tumor by administering a disclosed in situ gel-forming composition that comprises a chemotherapy agent. Examples of cancer include, but are not limited to, lung cancer, cancer of the peritoneum, hepatocellular cancer, gastric or stomach cancer including gastrointestinal cancer, pancreatic cancer, glioblastoma, cervical cancer, ovarian cancer, liver, cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, brain, and associated metastases.

As another illustrative example, the method relates to the vaccination of a subject using an in situ gel-forming composition that comprises a protein or peptide. Thus, the present disclosure provides a method of vaccinating a subject, comprising administering to the subject an effective amount of in situ gel-forming composition as described herein, where the biologically active agent will induce an immune response suitable for vaccination.

As another illustrative example, the method relates to the treatment of a disease of a subject using an in situ gel-forming composition that comprises an antibody, immune cell, nucleic acids, CRISPR components, CarT cells.

The treatment can be a single injection/application or multiple injections/applications that are separated by a period of time. A composition can be injected subcutaneously, intra-dermally or intra-muscularly, as several examples.

Compositions disclosed herein provide controlled release of at least one bioactive agent. Controlled release by disclosed compositions may comprise release of an effective amount of a bioactive agent, which may comprise from about 0.3 percent of total weight of the bioactive agent in the composition to about 50 percent of total weight of the bioactive agent in the composition per day, from about 0.5 percent of total weight of the bioactive agent in the composition to about 50 percent of total weight of the bioactive agent in the composition per day, from about 1 percent to about 40 percent, from about 5 percent to about 30 percent, from about 1 percent to about 20 percent, from about 5 percent to about 15 percent, from about 1 percent to about 10 percent, from about 0.5 percent to about 20 percent, or from about 0.5 percent per day, from about 1.0 percent per day, from about 2.0 percent per day, from about 3.0 percent per day, from about 4.0 percent per day, from about 5.0 percent per day, from about 6.0 percent per day, from about 7.0 percent per day, from about 8.0 percent per day, from about 9.0 percent per day, from about 10.0 percent per day, from about 12.0 percent per day, from about 15.0 percent per day, from about 18.0 percent per day, from about 20.0 percent per day, from about 25.0 percent per day, from about 30.0 percent per day, from about 35.0 percent per day, from about 40.0 percent per day, from about 45.0 percent per day, or from about 50% per day, and all ranges and percentages thereinbetween.

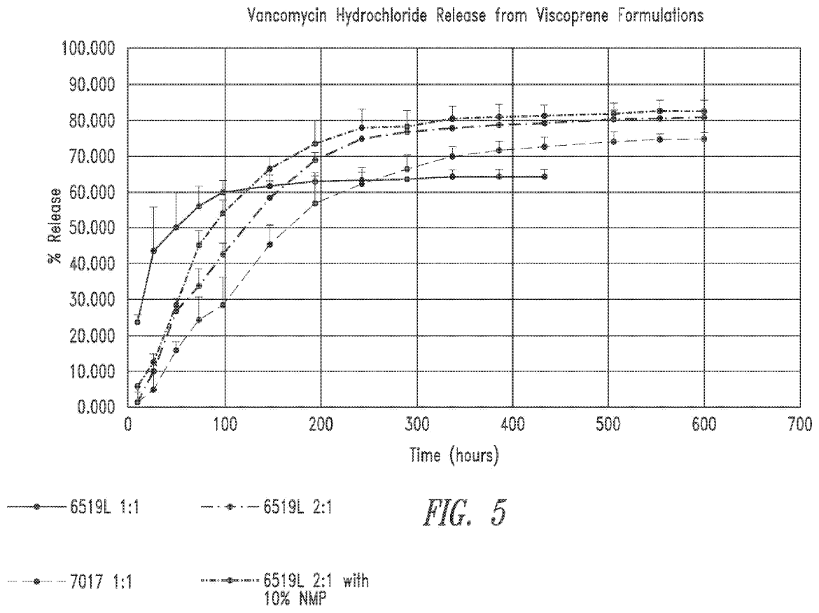

In an aspect, a disclosed composition herein releases at least one bioactive agent in a range of about 5 percent of total weight of the bioactive agent in the composition to about 50 percent of total weight of the bioactive agent in the composition in a selected period of time after administration of the composition. For example, a disclosed composition delivers an effective amount of a composition, which may comprise from about 5 percent of total weight of the bioactive agent in the composition to about 50 percent of total weight of the bioactive agent in the composition in a time period of 0.5 day, 1 day, of 2 days, of 3 days, of 4 days, of 5 days, of 6 days, of 7 days, of 8 days, of 9 days, of 10 days, of 11 days, of 12 days, of 13 days, of 14 days, of 15 days, of 20 days, or longer, with lesser amounts of the at least one bioactive agent continuing to be delivered by the disclosed composition for a longer period of time, for example, until about 80% to 90% of the absorbable polymer has been absorbed or is degraded. For example, a disclosed composition administers an effective amount of at least one bioactive agent in an amount between about 10 percent of total weight of the bioactive agent in the composition to about 40 percent of total weight of the bioactive agent in the composition in seven days after administration, with substantially the remainder of the bioactive agent released in about 21 days. For example, a disclosed composition administers an effective amount of at least one bioactive agent in an amount between about 20 percent of total weight of the bioactive agent in the composition to about 60 percent of total weight of the bioactive agent in the composition in 10 days after administration, with the remainder of the bioactive agent released for a longer time period. For example, a disclosed composition administers an effective amount of the majority of the weight of the total amount of at least one bioactive agent in the composition in less than 10 days after administration, with substantially the remainder of the bioactive agent release in lesser amounts for at least 10 following days. See FIG. 5 for release by exemplary compositions.

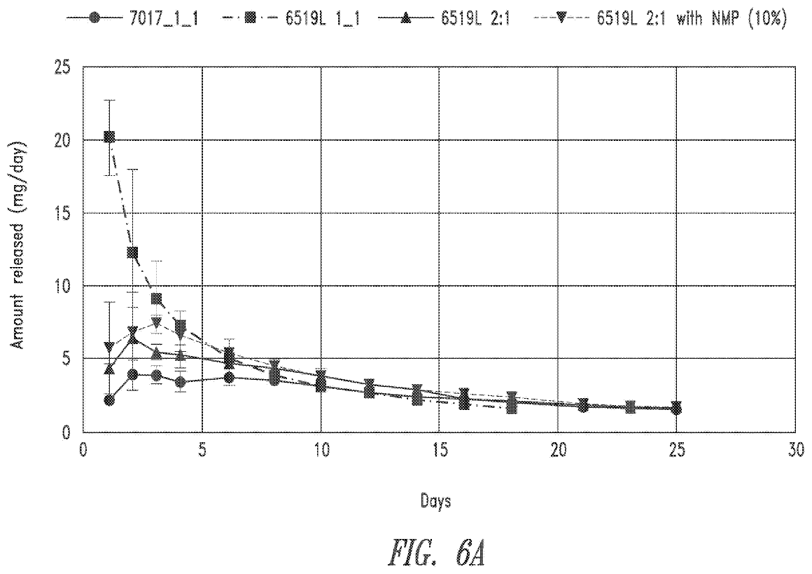

A composition disclosed herein provides an effective amount of at least one bioactive agent to a subject in need thereof in a controlled release manner. A controlled release manner may comprise a constant amount released of at least one bioactive agent, or may comprise a release of a larger bolus of at least one bioactive agent, followed by a period of a constant amount of release, or may comprise a release of a larger bolus of at least one bioactive agent, followed by a period of a steady amount of release, followed by a decreasing amount released per day as the absorbable polymer is absorbed or degraded. For example, a disclosed composition delivers or releases from about 0.5 to about 5 mg per day from about day 6 to about day 20 after administration to the subject. The disclosed composition may deliver more or fewer mg per day in the period between day 0 and day 6. The disclosed composition may deliver fewer mg per day in the period after day 20. For example, see FIG. 6C for release by exemplary compositions. As is understood by those of skill in the art, amounts released may be measured in grams, micrograms, nanograms, picograms or other amounts depending on the amount of bioactive agent and composition. As used herein, “release” and “deliver” are used interchangeably to describe bioactive agent leaving the administered composition.

A composition disclosed herein provides an effective amount of at least one bioactive agent to a subject in need thereof in a controlled release manner. For example, controlled release of an amount of at least one bioactive agent/day per g composition administered may be constant, or may vary after administration. For example, a disclosed composition delivers or releases from about 0.1 to about 20 mg per day per gram of composition for the first 2 days following administration, or from about 0.1 to about 5 mg per day per gram of composition for the first 2 days following administration, or from about 5 to about 10 mg per day per gram of composition for the first 2 days following administration, or from about 5 to about 20 mg per day per gram of composition for the first 2 days following administration, after which the amount released decreases per day per gram of composition administered as the absorbable polymer is absorbed or degraded. For example, a disclosed composition may release fewer mg of bioactive agent per day post administration per gram of composition after an initial period post administration. For example, a composition may release between 1-5 mg of bioactive agent per day per gram composition from day 6 to 20 post administration. For example, see FIG. 6A for release by exemplary compositions. As is understood by those of skill in the art, amounts released may be measured in grams, micrograms, nanograms, picograms or other amounts depending on the amount of bioactive agent and composition.

A composition disclosed herein provides an effective amount of at least one bioactive agent to a subject in need thereof in a controlled release manner. For example, controlled release of a concentration per day of at least one bioactive agent administered may be constant or may vary after administration. For example, a disclosed composition delivers or releases from about 0.1 to about 20 mg per mL per day for the first 2 days following administration, or from about 0.1 to about 5 mg per ml per day for the first 2 days following administration, or from about 5 to about 10 mg per mL per day for the first 2 days following administration, or from about 5 to about 20 mg per mL per day for the first 2 days following administration, after which the amount released per mL per day decreases as the absorbable polymer is absorbed or degraded. For example, a disclosed composition may release fewer mg of bioactive agent per mL per day post administration after an initial period post administration. For example, a composition may release between 1-5 mg of bioactive agent per mL per day from day 6 to 20 post administration. For example, see FIG. 6B for release by exemplary compositions. As is understood by those of skill in the art, amounts released may be measured in grams, micrograms, nanograms, picograms or other amounts depending on the amount of bioactive agent and the volume of composition administered.

### General Comments

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

All references disclosed herein, including patent references and non-patent references, are hereby incorporated by reference in their entirety as if each was incorporated individually.

It is to be understood that the terminology used herein is for the purpose of describing specific embodiments only and is not intended to be limiting. It is further to be understood that unless specifically defined herein, the terminology used herein is to be given its traditional meaning as known in the relevant art.

Reference throughout this specification to “an aspect” and variations thereof means that a particular feature, structure, or characteristic described in connection with the aspect is included in at least one embodiment. Thus, the appearances of the phrases “in an aspect” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents, i.e., one or more, unless the content and context clearly dictates otherwise. It should also be noted that the conjunctive terms, “and” and “or” are generally employed in the broadest sense to include “and/or” unless the content and context clearly dictates inclusivity or exclusivity as the case may be. Thus, the use of the alternative (e.g., “or”) should be understood to mean either one, both, or any combination thereof of the alternatives. In addition, the composition of “and” and “or” when recited herein as “and/or” is intended to encompass an embodiment that includes all of the associated items or ideas and one or more other alternative embodiments that include fewer than all of the associated items or ideas.

Unless the context requires otherwise, throughout the specification and claims that follow, the word “comprise” and synonyms and variants thereof such as “have” and “include”, as well as variations thereof such as “comprises” and “comprising” are to be construed in an open, inclusive sense, e.g., “including, but not limited to.” The term “consisting essentially of” limits the scope of a claim to the specified materials or steps, or to those that do not materially affect the basic and novel characteristics of the claimed disclosure.

Any headings used within this document are only being utilized to expedite its review by the reader, and should not be construed as limiting the disclosure or claims in any manner. Thus, the headings and Abstract of the Disclosure provided herein are for convenience only and do not interpret the scope or meaning of the embodiments.

Where a range of values is provided herein, it is understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

For example, any concentration range, percentage range, ratio range, or integer range provided herein is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Expressly incorporated by reference are U.S. Patent publication documents 2003/162940, 2009/233887, 2009/291925, 2010/0255016, 2012/0077823, 2012/0208789, 2013/0095087, 2013/3331537, 2014/046019, 2015/0157726 and 2015/0265643. Such documents may be incorporated by reference for the purpose of describing and disclosing, for example, materials and methodologies described in the publications, which might be used in connection with the presently described disclosure. The publications discussed above and throughout the text are provided solely for their disclosure prior to the filing date of the present application. Nothing herein is to be construed as an admission that the inventors are not entitled to antedate any referenced publication by virtue of prior disclosure. Applicants reserve the right to physically incorporate into this specification any and all materials and information from any such patents, publications, scientific articles, web sites, electronically available information, and other referenced materials or documents.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

Furthermore, the written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

Other nonlimiting embodiments are within the following claims. The patent may not be interpreted to be limited to the specific examples or nonlimiting embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

Further illustrations of the present disclosure are provided by the following examples:

### Example 1

### Synthesis of 7017: Copolymer Containing 58 wt. % d,l-Lactide, 30 wt. % PEG 400, and 12 wt. % Glycolide, Interlinked at 1.0:0.8 Initiator:HMDI

81.9 g PEG 400 was added to glass kettle with an oil bath set to 100° C. 31.9 g glycolide and 159.6 g DL-lactide was added and stirred at 120 RPM. Once fully mixed, 574 μl of the catalyst solution (0.2M Tin (II)-2-ethylhexanoate, 96% in toluene) was added. The solution was heated at 140° C. for 14.5 hours at which time the heat was removed. Once the temperature of the solution was below 40° C., the stirring speed was set to 120 RPM and 27.5 g diisocyanatohexane was added. The temperature was increased to 100° C. with stirring conditions maintained at 120 RPM for 1 hour at which time the temperature was decreased to 60° C. Tetrahydrofuran (293.8 g) was then added to the mixture and stirred until all the polymer was dissolved at which time Isopropanol (6800 μl) was added and allowed to stir for 1 hour at which time the heat was removed. The polymer was then purified in 100 g aliquots using a combination of 700 g refrigerator chilled water and 300 g of deionized ice mixed in an industrial blender. The purified solution was then dried in an 80° C. vacuum oven for greater than 14 hours. The average Mw of the polymer, as measure by GPC (THF as solvent) was 15,671 Da.

### Example 2

### Synthesis of 6519L: Copolymer Containing 50 wt. % d,l-Lactide, 40 wt. % PEG 400, and 10 wt. % Glycolide, Interlinked at 1.0:0.8 Initiator:HMDI

105.8 g PEG 400 was added to a glass kettle with an oil bath set to 100° C. for one hour. 26.8 g glycolide and 132.2 g DL-lactide was added and stirred at 120 RPM. Once fully mixed, 477 μl of the catalyst solution (0.2M Tin (II)-2-ethylhexanoate, 96% in toluene) was added. The solution was heated at 140° C. for 14.5 hours at which time the heat was removed. Once the temp of the solution was below 40° C., 35.6 g diisocyanatohexane was added and stirred at 120 RPM. The temperature was increased to 100° C. with stirring conditions maintained at 120 RPM for 1 hour at which time the temperature was decreased to 60° C. Tetrahydrofuran (300.0 g) was then added to the mixture and stirred until all the polymer was dissolved at which time Isopropanol (8800 μl) was added and allowed to stir for 1 hour at which time the heat was removed and cooled to room temperature. The polymer was then purified in 75-100 g aliquots using a combination of 700 g refrigerator chilled water and 300 g of deionized ice mixed in an industrial blender. The purified solution was then dried in an 80° C. vacuum oven for greater than 14 hours. The average Mw of the polymer, as measure by GPC (THF as solvent) was 11,167 Da.

### Example 3

### Synthesis of 6519M: Copolymer Containing 50 wt. % d,l-Lactide, 40 wt. % PEG 400, and 10 wt. % Glycolide, Interlinked at 1.0:1.2 Initiator:HMDI

105.8 g PEG 400 was added to a glass kettle with an oil bath set to 100° C. under nitrogen conditions for one hour. 26.6 g glycolide and 133.4 g DL-lactide was added and stirred at 120 RPM. Once fully mixed, 477 μl of the catalyst solution (0.2M Tin (II)-2-ethylhexanoate, 96% in toluene) was added. The solution was heated at 140° C. for 14.5 hours at which time the heat was removed. Once the temp of the solution was below 40° C., 53.7 g diisocyanatohexane was added and stirred at 120 RPM. The temperature was increased to 100° C. with stirring conditions maintained at 120 RPM for 1 hour at which time the temperature was decreased to 60° C. Tetrahydrofuran (302.9 g) was then added to the mixture and stirred until all the polymer was dissolved at which time Isopropanol (13200 μl) was added and allowed to stir for 1 hour at which time the heat was removed and cooled to room temperature. The polymer was then purified in 75-100 g aliquots using a combination of 700 g refrigerator chilled water and 300 g of deionized ice mixed in an industrial blender. The purified solution was then dried in an 80° C. vacuum oven for greater than 14 hours. The average Mw of the polymer, as measure by GPC (THF as solvent) was 21,816 Da.

### Example 4

### Injectability and Rheology Characterization of Polymer Compositions

Variations of the polymer compositions 6519L, 6519M and 7017, varied diluent (PEG 400 and DMF ratios, and viscosity modifiers 5% w/w polyglycolide polymer powder (referred to herein as A6) and mixed. Viscosity analysis was performed on the Anton Paar MCR 301 rheometer. Each specimen was tested using a shear rate frequency sweep from 0.1 s-1 to 100 s-1, and at a temperature of 25° C., with viscosity data reported [Table 1] as the average from 1 s-1 to 100 s-1. Minimum viscosity readings resulted from 6519M at a 1:2 ratio (w/w) polymer/PEG 400 with 10% (w/w) DMF at about 1,200 cP. Maximum viscosity readings resulted from 6519M at a 2:1 ratio (w/w) polymer/PEG 400 and a maximum viscosity value of about 474,000 cP. Injectability testing was performed with some of the same compositions with approximately 0.5 ml of each composition loaded into a 1 ml syringe with or without a needle (18G, 21G, or 23G). The syringe plungers were compressed using Material Testing System (MTS) Synergie 200 with 1 kN Load Cell with a test speed of 0.1 mL/s, which corresponds to 5.76 mm/s for a 1 mL syringe. Injection forces [Table 2] of the various compositions without test failure (due to plunger deformation or needle failure) ranged from 1-26 N without a needle, 25-265 N with an 18 Ga. needle, 97-170 N with a 21 Ga. needle, and 148-215 N with a 23 Ga. needle.

TABLE 1

| Disclosed | Mass ratio of | | Mass ratio of Polymer/ | Viscosity (cP) | | |
|---|---|---|---|---|---|---|
| compositions | Polymer:PEG-400 | Diluent | PEG-400:Diluent | Average | Std. Dev. | Replicates |
| 6519M_1:1_10% DMF | 1:1 | DMF | 9:1 | 14,452 | 392 | 4 |
| 6519M_1:1_5% A6 | 1:1 | A6 | 19:1 | 71,345 | 1,254 | 4 |
| 6519M_1:1_None | 1:1 | None | N/A | 65,729 | 938 | 6 |
| 6519M_1:2_10% DMF | 1:2 | DMF | 9:1 | 2,508 | 4 | 4 |
| 6519M_1:2_5% A6 | 1:2 | A6 | 19:1 | 11,321 | 58 | 4 |
| 6519M_1:2_None | 1:2 | None | N/A | 8,557 | 153 | 6 |
| 6519M_2:1_10% DMF | 2:1 | DMF | 9:1 | 61,616 | 577 | 4 |
| 6519M_2:1_None | 2:1 | None | N/A | 473,874 | 14,328 | 7 |
| 6519L_1:1_10% DMF | 1:1 | DMF | 9:1 | 3,662 | 533 | 4 |
| 6519L_1:1_5% A6 | 1:1 | A6 | 19:1 | 20,486 | 136 | 4 |
| 6519L_1:1_None | 1:1 | None | N/A | 13,121 | 146 | 6 |
| 6519L_1:2_10% DMF | 1:2 | DMF | 9:1 | 1,197 | 10 | 4 |
| 6519L_1:2_5% A6 | 1:2 | A6 | 19:1 | 3,982 | 19 | 4 |
| 6519L_1:2_None | 1:2 | None | N/A | 3,458 | 47 | 7 |
| 6519L_2:1_10% DMF | 2:1 | DMF | 9:1 | 22,973 | 395 | 4 |
| 6519L_2:1_None | 2:1 | None | N/A | 159,147 | 3,918 | 7 |
| 7017_1:1_10% DMF | 1:1 | DMF | 9:1 | 5,856 | 42 | 4 |
| 7017_1:1_None | 1:1 | None | N/A | 28,242 | 644 | 7 |
| 7017_1:2_10% DMF | 1:2 | DMF | 9:1 | 1,299 | 4 | 4 |
| 7017_1:2_None | 1:2 | None | N/A | 4,400 | 37 | 7 |
| 7017_2:1_10% DMF | 2:1 | DMF | 9:1 | 29,803 | 479 | 4 |
| 7017_2:1_None | 2:1 | None | N/A | 222,606 | 1,899 | 7 |

TABLE 2

| | Injection Force (N) | | | |
|---|---|---|---|---|
| Disclosed | No | 18G | 21G | 23G |
| compositions | Needle | Needle | Needle | Needle |
| 6519M_1:1_10% DMF | 2.794 | 88.512 | Not | Not |
| | | | measured | measured |
| 6519M_1:1_5% A6 | 6.049 | 223.167 | Not | Not |
| | | | measured | measured |
| 6519M_1:1_None | 5.243 | 212.953 | Not | Not |
| | | | measured | measured |
| 6519M_1:2_10% DMF | 1.793 | Not | 97.418 | Not |
| | | measured | | measured |
| 6519M_1:2_5% A6 | 3.648 | 69.01 | Not | Not |
| | | | measured | measured |
| 6519M_1:2_None | 1.293 | 50.614 | 169.604 | Not |
| | | | | measured |
| 6519M_2:1_10% DMF | 5.429 | 231.966 | Not | Not |
| | | | measured | measured |
| 6519M_2:1_None | 26.133 | 265.129 | Not | Not |
| | | | measured | measured |
| 6519L_1:1_10% DMF | 1.932 | 25.397 | 131.118 | Not |
| | | | | measured |
| 6519L_1:1_5% A6 | 4.952 | 130.019 | Not | Not |
| | | | measured | measured |
| 6519L_1:1_None | 3.939 | 107.477 | Not | Not |
| | | | measured | measured |
| 6519L_1:2_10% DMF | 1.671 | Not | 51.669 | 157.736 |
| | | measured | | |
| 6519L_1:2_5% A6 | 1.435 | 26.349 | 143.606 | Not |
| | | | | measured |
| 6519L_1:2_None | 1.127 | 24.724 | 123.81 | 214.387 |
| 6519L_2:1_10% DMF | 5.262 | 145.294 | Not | Not |
| | | | measured | measured |
| 6519L_2:1_None | 12.146 | 251.918 | Not | Not |
| | | | measured | measured |
| 7017_1:1_10% DMF | 2.413 | 41.643 | 161.688 | Not |
| | | | | measured |
| 7017_1:1_None | 2.905 | 147.968 | Not | Not |
| | | | measured | measured |
| 7017_1:2_10% DMF | 1.685 | Not | 57.173 | 148.051 |
| | | measured | | |
| 7017_1:2_None | Not | Not | Not | Not |
| | measured | measured | measured | measured |
| 7017_2:1_10% DMF | 4.752 | 163.908 | Not | Not |
| | | | measured | measured |
| 7017_2:1_None | 15.439 | 250.358 | Not | Not |
| | | | measured | measured |

### Example 5

### Addition of a Biologically Active Agent to the Polymer/Solvent Compositions

About 7.13 g of the polymer/PEG-400 mixture was added to a mixing vessel. About 0.375 g vancomycin (used as a representative biologically active agent) was added to the mixture. The resultant composition was mixed using a mixer until the material was dissolved or uniformly dispersed in the composition.

### Example 6

### Addition of a Biologically Active Agent to the Polymer Solvent1/Solvent2 Compositions

About 6.4 g of the polymer/PEG-400 mixture (6519L/PEG-400 at a mass ratio of 2:1) was added to a mixing vessel. About 0.72 g N-methylpyrrolidone (NMP) was added to the composition. The resultant composition was mixed using a mixer until a homogeneous solution was obtained. About 0.375 g vancomycin (used as a representative biologically active agent) was added to the mixture. The resultant composition was mixed using a mixer until the material was dissolved or uniformly dispersed in the composition.

### Example 7

### Exemplary Polymer Compositions and Characterization Thereof

Table 3 provides further exemplary compositions of the present disclosure. In Table 1, compositions are prepared from three base polymers (6519L, 6519M and 7017) according to the present disclosure, and compositions thereof are described in terms of viscosity and injection force (max) for different polymer/diluent ratios, where DMF (dimethylformamide) is the diluent. The base polymers 6519L, 6519M and 7017 are variations of d,l lactide (65-70 mol %), glycolide (16-18 mol %), and PEG 400 (13-20 mol %) with diisocyanate interlinker.

Composition viscosity screenings were performed on an Anton Parr MCR 300 rheometer using a CP-25 measuring system and a frequency sweep of 1-100 s-1 at 25° C. The force needed to eject a composition from a needle, where the needle has either an 18 gauge, 21 gauge or 23 gauge needle, is provided for several of the compositions. Composition injectability testing was performed on a Synergie MTS with 1 ml syringes with 18-23 Ga needles tested at a compression rate of 5.76 mm/sec.

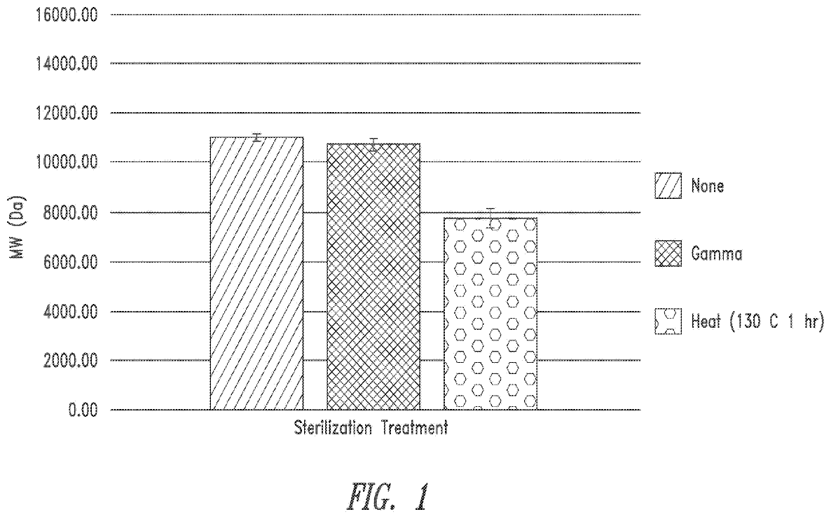

In addition to the characterization in Table 1, selected data for selected compositions is provided in FIGS. 1, 2 and 3.

FIG. 1 shows effect of sterilization on the molecular weight of polymers incorporated into compositions of the present disclosure (n=3). Compositions were exposed to gamma radiation sterilization in the specified dosage range of 25-30 kGy (Steric) and compared to unsterilized controls.

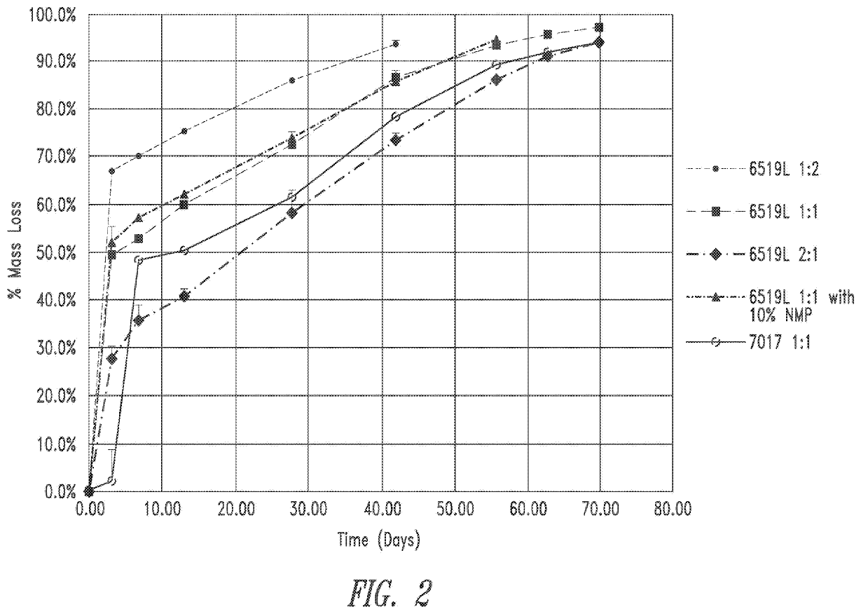

FIG. 2 shows mass loss degradation profiles for compositions of the present disclosure, as measured over 70 days (n=4). Five (5) compositions were aliquoted into 0.5 g samples for in vitro testing (mass loss, composition, and MW) over a course of up to two (2) months in accordance with ASTM 1635-049.

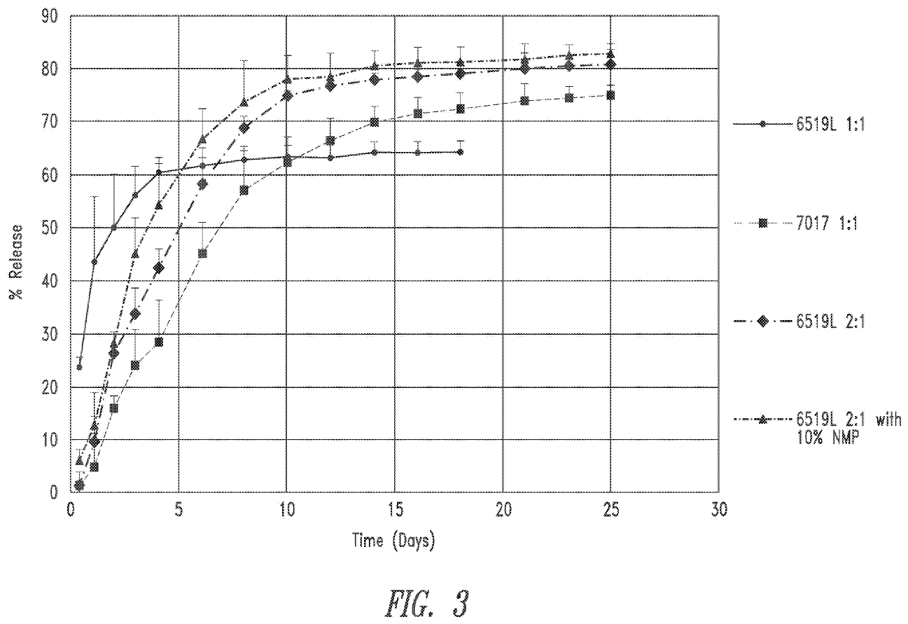

FIG. 3 shows release of a hydrophilic drug (vancomycin hydrochloride) from compositions of the present disclosure, as measured over 25 days (n=3). The release of vancomycin hydrochloride (Sigma Aldrich 1709007) (5% w/w) was evaluated in four (4) compositions by HPLC analysis (Waters HPLC with Symmetry C18 5 um HPLC column) over a duration of approximately 3 weeks in 1 g composition aliquots in PBS (pH=7.4).

TABLE 3

| Composition | | Viscosity (cP) | Injection Force Max (N) | | | |
|---|---|---|---|---|---|---|
| Base | Polymer/ | at 1.1 s⁻¹ | No | 18G | 21G | 23G |
| Polymer | PEG/Diluent | (min of n = 4) | Needle | Needle | Needle | Needle |
| 6519L | 1:2 | 3489 ± 41 | 1.13 | 24.72 | 123.81 | 214.39 |
| | 1:2 w/10% DMF | 1198 ± 17.1 | 1.67 | | 51.67 | 157.74 |
| | 1:1 | 13150 ± 183 | 3.94 | 107.48 | | |
| | 1:1 w/10% DMF | 4825 ± 2713 | 1.93 | 25.40 | 131.12 | |
| | 2:1 | 159714 ± 3904 | 12.15 | 251.92 | | |
| | 2:1 w/10% DMF | 23000 ± 424 | 5.26 | 145.29 | | |
| 6519M | 1:2 | 8622 ± 224 | 1.29 | 50.61 | 169.60 | |
| | 1:2 w/10% DMF | 2508 ± 9.6 | 1.79 | | 97.42 | |
| | 1:1 | 65937 ± 944 | 5.24 | 212.95 | | |
| | 1:1 w/10% DMF | 14475 ± 395 | 2.79 | 88.51 | | |
| | 2:1 | 494571 ± 13240 | 26.13 | 265.13 | | |
| | 2:1 w/10% DMF | 61925 ± 591 | 5.43 | 231.97 | | |
| 7017 | 1:2 | 4413 ± 43 | | | | |
| | 1:2 w/10% DMF | 1305 ± 10 | 1.69 | | 57.17 | 148.05 |
| | 1:1 | 28283 ± 674 | 2.91 | 147.97 | | |
| | 1:1 w/10% DMF | 5910 ± 110 | 2.41 | 41.64 | 161.69 | |
| | 2:1 | 226143 ± 1952 | 15.44 | 250.36 | | |
| | 2:1 w/10% DMF | 29875 ± 443 | 4.75 | 163.91 | | |

In terms of handling (viscosity and injectability), a limit of 150N or less under the testing conditions presents several options up to a 21G needle (Table 3). Flexibility of sterilization is important for product development and commercialization. Although sterilization is dependent upon the loaded agent stability, GPC results (FIG. 1) support the stability when exposed to gamma sterilization (25 kGy) which was not the case for applied heat (130° C. for 1 hr). Bioburden results from Steris (not shown) were also negative.

Composition degradation (FIG. 2) demonstrated a range of 5-9 weeks to achieve >90% mass loss. High initial mass loss % can be attributed to the hydrophilic diluent exiting the composition first when in contact with an aqueous environment. GPC testing (data not shown) supported the degradation of the material in similar trends to the mass loss. Although high mass loss was observed initially, release studies in FIG. 3 shows a lower initial slope for the hydrophilic agent release but follows a similar trend in that the greater the diluent amount, the greater the percentage of agent release, therefore allowing for tailored release product design.

### Example 8

### Exemplary Bioactive Agent Release Study

The disclosed compositions of Table 4 were used in the release study, and made as described above.

| | Ratio | % NMP (w/w %) | % Vancomycin (w/w %) |
|---|---|---|---|
| | of Polymer | of final | of final |
| Polymer | to PEG 400 | composition | composition |
| 7017 | 1:1 | 0 | 5% |
| 6519L | 1:1 | 0 | 5% |
| 6519L | 2:1 | 0 | 5% |
| 6519L | 2:1 | 10% | 5% |

### In Vitro Release Study Aliquots

Compositions were placed in 37° C. incubator for 1-2 hours depending on when they were removed for aliquots. Each composition was aliquoted to 1.0±0.02 g in 20 ml glass vials at n=3 sampling. Vials were nitrogen capped and taped and placed back in the incubator as they were complete to allow for the individual composition aliquots to settle under gentle oscillation. After overnight (>12 h) oscillation, samples were removed from the incubator and allowed to cool to room temperature for approximately one hour. Ten (10) mL of prepared PBS equilibrated to 37° C. was applied to each vial and they were taped and placed back in the incubator. At time points 9 h, 26 h, 49 h, 73 h, 97 h, 146.5 h, 193.5 h, 241.5 h, 289 h, 337 h, 385 h, 433 h, 505 h, 553 h, and 601 h, PBS was collected for HPLC analysis and the ten (10 ml) of PBS equilibrated to 37° C. was replenished to each vial. If collected release study PBS samples were not used the same day, the vials were generally stored under refrigerated (2-8° C.) conditions. Following completion of the release study for each respective test group, remaining composition vials were dried under RT vacuum conditions for further analysis purposes. Gel Permeation Chromatography (GPC) via HPLC

The following GPC testing was performed according to TM 18-00-036 GPC (Waters THF), a known method. Compositions post vancomycin and NMP (if applicable) were tested, including partially degraded samples following conclusion of the release study.

Sample prep included approximately 12.0 mg sample material dissolved in tetrahydrofuran (THF) solvent to final sample concentration of 4 mg/ml. When the polymer was fully dissolved (under mechanical shaking), the sample was filtered through 0.45 um filter. An injection volume of 40 ul was used for a run time of 35 min, at a flow rate of 1 ml/min. Resultant peaks on chromatogram were analyzed for Mn, Mw, Mp, MZ, and PDI (Mw/Mn).

### Rheological Analysis

The following rheological testing was performed. Compositions post vancomycin and NMP (if applicable) were tested, including partially degraded samples following conclusion of the release study. Disclosed composition samples were applied to 25° C. surface of an Anton Paar MCR 301 Rheometer with a CP-25-1 Measuring system (25 mm cone on plate with 1° angle). A flow curve program with shear rate (Y) sweep from 1-1000 1/s was used.

### HPLC

One (1) gram aliquots of each disclosed composition with n=3 sampling as described above were tested at respective time points according to the following procedure. Collected PBS samples were filtered through 0.45 μm PP syringe filter and remaining buffer gently aspirated to not disturb the composition aliquot. PBS equilibrated to 37° C. was replenished in each vial at a volume of 10 ml, pipetted along the size of the vial to minimize composition aliquot disturbance. Samples were then placed back in 37° C. incubator. Collected PBS samples were run on Waters HPLC with Symmetry C18 5 um HPLC column using the method developed for vancomycin hydrochloride. The isocratic method utilized 89% water and 11% acetonitrile (both containing 0.1% trifluoroacetic acid) at a flow rate of 1.0 mL per minute. Detection was performed via a UV-Vis detector at 205 nm. The run time was 10 minutes with the vancomycin eluting around 6.5 minutes. Injection volumes were adjusted as necessary for analysis in regard to the limits of the standard curve. Note: if collected samples were not immediately run following collection, then they were stored at 4° C. for later testing. When release curve had plateaued, a small amount (˜10-15 mg) was removed from each vial for GPC analysis and the remaining samples were dried under RT vacuum conditions until constant weight achieved.

Extractions were performed on the remaining samples according to the following procedure. Acetonitrile (ACN) (2 ml) was added to each vial and placed on shaker for approximately two (2) hour. DI water (2 ml) was added to each vial and vortexed for 1 minute. Eppendorf tubes were filled with approximately 1.5 ml of each vial and centrifuged for ten (10) minutes at 13,000 RPM in a centrifuge, Biofuge 13. Supernatants were then run on the HPLC using the vancomycin hydrochloride method and vancomycin content was determined. Glass scintillation vials were rinsed with acetone and place under vacuum to obtain empty vial weights and used to calculate the amount of gel remaining after the release study was concluded.

### Gel Permeation Chromatography

GPC results from Table 4 and Table 5 indicated that there was not a significant decrease in molecular weight due to the addition of our vancomycin and NMP additives based on t-test analysis performed in Minitab statistical software. In other words, p>0.05 for all cases when comparing pre-mixed versus post-mixed compositions (Table 5). GPC results from degraded release samples indicated a ˜30% decrease in 6519L (1:1 and 2:1) compositions and ˜20% in the 7017 composition.

TABLE 4
GPC Summary Results for a) Initial compositions without agent
or NMP (when applicable), b) Compositions following addition
(mixing) of agent and c) Compositions following the completion
of their respective final degradation study time points

| | | |
|---|---|---|
| Initial | Initial | Degraded |
| (Pre-mixing) | (Post-mixing) | (Post Release Study) |

Mn (Daltons)

| | | | |
|---|---|---|---|
| 6519L 1:1 | 9208 ± 105 | 8941 ± 260 | 6034 ± 118 |
| 7017 1:1 | 9768 ± 949 | 9406 ± 190 | 7539 ± 52 |
| 6519L 2:1 | 8822 ± 223 | 8965 ± 549 | 5987 ± 135 |
| 6519L | 8805 ± 240 | 9124 ± 228 | 5856 ± 238 |
| w/10% NMP | | | |

Mw (Daltons)

| | | | |
|---|---|---|---|
| 6519L 1:1 | 13566 ± 158 | 13323 ± 185 | 8797 ± 79 |
| 7017 1:1 | 16324 ± 606 | 15968 ± 68 | 11787 ± 164 |
| 6519L 2:1 | 13280 ± 108 | 13464 ± 215 | 8610 ± 127 |
| 6519L | 13272 ± 221 | 13358 ± 204 | 8368 ± 337 |
| w/10% NMP | | | |

Mp (Daltons)

| | | | |
|---|---|---|---|
| 6519L 1:1 | 12522 ± 151 | 12376 ± 88 | 7933 ± 54 |
| 7017 1:1 | 14718 ± 375 | 14517 ± 66 | 10541 ± 146 |
| 6519L 2:1 | 12339 ± 43 | 12719 ± 372 | 7788 ± 135 |
| 6519L | 12763 ± 456 | 12502 ± 105 | 7582 ± 261 |
| w/10% NMP | | | |

PDI

| | | | |
|---|---|---|---|
| 6519L 1:1 | 1.47 ± 0.000 | 1.49 ± 0.023 | 1.46 ± 0.019 |
| 7017 1:1 | 1.68 ± 0.097 | 1.70 ± 0.035 | 1.56 ± 0.015 |
| 6519L 2:1 | 1.51 ± 0.027 | 1.50 ± 0.078 | 1.44 ± 0.013 |
| 6519L | 1.47 ± 0.034 | 1.46 ± 0.027 | 1.43 ± 0.003 |
| w/10% NMP | | | |

TABLE 5
Statistical GPC Comparison of Initial compositions prior to agent
loading (mixing) and compositions following agent loading (mixing)

| | | Variance | | T-test | |
|---|---|---|---|---|---|
| | | P | | P | |
| | | value | Result | value | Result |
| 6519L | Mn | 0.279 | variances equivalent | 0.175 | means equivalent |
| 1:1 | Mw | 0.847 | variances equivalent | 0.159 | means equivalent |
| | MP | 0.506 | variances equivalent | 0.221 | means equivalent |
| | PDI | 0.001 | Variances not | 0.260 | means equivalent |
| | | | equivalent | | |
| 7017 | Mn | 0.077 | variances equivalent | 0.552 | means equivalent |
| 1:1 | Mw | 0.025 | Variances not | 0.369 | means equivalent |
| | | | equivalent | | |
| | MP | 0.06 | variances equivalent | 0.411 | means equivalent |
| | PDI | 0.234 | variances equivalent | 0.744 | means equivalent |
| 6519L | Mn | 0.283 | variances equivalent | 0.698 | means equivalent |
| 2:1 | Mw | 0.404 | variances equivalent | 0.254 | means equivalent |
| C2-3 | MP | 0.027 | Variances not | 0.154 | means equivalent |
| | | | equivalent | | |
| | PDI | 0.209 | variances equivalent | 0.988 | means equivalent |
| 6519L | Mn | 0.949 | variances equivalent | 0.171 | means equivalent |
| 2:1 | Mw | 0.920 | variances equivalent | 0.647 | means equivalent |
| C2-4 | MP | 0.100 | variances equivalent | 0.388 | means equivalent |
| | PDI | 0.789 | variances equivalent | 0.733 | means equivalent |

### Rheological Analysis

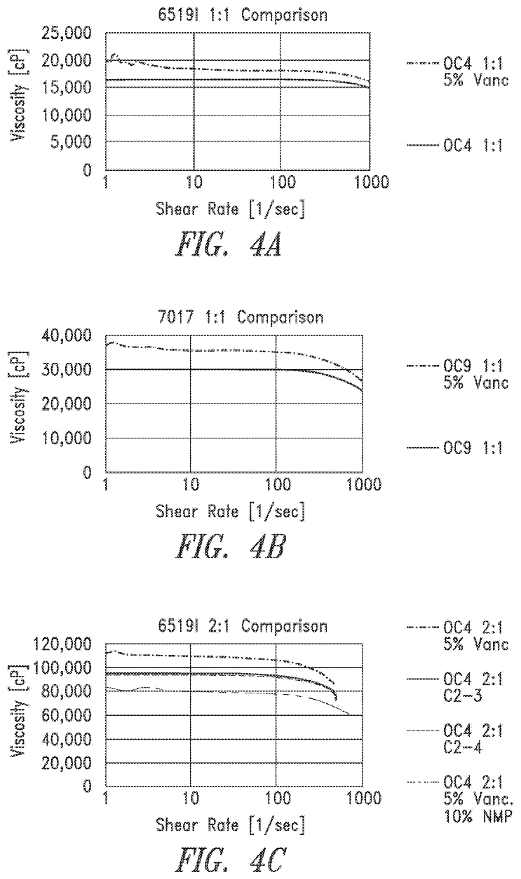

Rheological analysis shown in FIGS. 4A-C and Table 5 below for the compositions overall demonstrated an average of 15.0% increase in viscosity when loaded with 5% (w/w) Vancomycin loading for the three compositions. The addition of the 10% (w/w) NMP within the 6519L composition resulted in a 26.9% decrease in viscosity. FIG. 4A-C show the primarily Newtonian rheological response of all compositions from 1-100 1/sec with shear thinning region from 100-1000 1/sec.

TABLE 5
Avg Viscosity (cP) at Select Shear Rates (n = 3)

| | Shear Rate (1/s) | | | | | |
|---|---|---|---|---|---|---|
| Sample | 1 | | 10 | | 105 | |
| Reference | Mean | Std. Dev. | Mean | Std. Dev. | Mean | Std. Dev. |
| 6519L 1:1 | 16,467 | 351 | 16,400 | 300 | 16,333 | 252 |
| 6519L 1:1, 5% | 20,133 | 1,457 | 18,333 | 351 | 18,000 | 265 |
| Vancomycin | | | | | | |
| 7017 1:1 | 30,233 | 503 | 30,200 | 361 | 29,933 | 404 |
| 7017 1:1, 5% | 36,767 | 666 | 35,567 | 874 | 34,900 | 985 |
| Vancomycin | | | | | | |
| 6519L 2:1 C2-3 | 94,300 | 0 | 94,900 | 1,212 | 93,267 | 1,069 |
| 6519L 2:1 C2-4 | 94,233 | 2,892 | 94,067 | 2,875 | 92,400 | 2,663 |
| 6519L 2:1, 5% | 112,333 | 4,041 | 109,667 | 2,082 | 106,000 | 1,732 |
| Vancomycin | | | | | | |
| 6519L 2:1, 5% | 83,467 | 9,613 | 80,200 | 9,500 | 77,567 | 9,274 |
| Vancomycin, | | | | | | |
| 10% NMP | | | | | | |

TABLE 6

| | | 6519L 1:1 | | 7017 1:1 | | 6519L 2:1 | | 6519L 2:1 with 10% NMP | |
|---|---|---|---|---|---|---|---|---|---|
| Days | Hours | Average | Std Dev | Average | Std Dev | Average | Std Dev | Average | Std Dev |
| 0.4 | 9 | 23.582 | 1.903 | 1.499 | 0.138 | 2.058 | 0.183 | 5.838 | 2.364 |
| 1.1 | 26 | 43.491 | 12.328 | 4.845 | 2.179 | 9.588 | 4.742 | 12.624 | 6.051 |
| 2.0 | 49 | 50.015 | 10.050 | 15.922 | 2.453 | 26.393 | 1.886 | 27.884 | 2.253 |
| 3.0 | 73 | 56.082 | 5.370 | 24.013 | 4.176 | 33.868 | 4.754 | 45.041 | 6.784 |
| 4.0 | 97 | 60.094 | 3.050 | 28.351 | 3.836 | 42.233 | 3.671 | 53.992 | 7.809 |
| 6.1 | 146.5 | 61.661 | 3.264 | 45.122 | 3.350 | 58.139 | 4.975 | 66.369 | 5.842 |
| 8.1 | 193.5 | 62.732 | 2.597 | 56.909 | 6.155 | 68.748 | 2.240 | 73.482 | 7.498 |
| 10.1 | 241.5 | 63.046 | 2.453 | 62.489 | 5.399 | 74.768 | 0.956 | 77.693 | 4.371 |
| 12.0 | 289 | 63.276 | 2.432 | 66.279 | 4.465 | 76.733 | 1.023 | 78.252 | 4.186 |
| 14.0 | 337 | 64.133 | 2.073 | 69.823 | 3.746 | 77.793 | 1.237 | 80.250 | 2.781 |
| 16.0 | 385 | 64.184 | 2.038 | 71.551 | 3.626 | 78.497 | 1.418 | 80.792 | 2.792 |
| 18.0 | 433 | 64.263 | 2.010 | 72.505 | 3.260 | 79.080 | 1.850 | 81.027 | 2.750 |
| 21.0 | 505 | N/A | N/A | 73.998 | 3.184 | 80.067 | 2.869 | 81.571 | 2.749 |
| 23.0 | 553 | N/A | N/A | 74.554 | 3.252 | 80.439 | 2.728 | 82.298 | 1.712 |
| 25.0 | 601 | N/A | N/A | 74.877 | 3.289 | 80.995 | 2.458 | 82.441 | 1.658 |

### Extraction

Vancomycin Percentage Recovery

| Composition | Avg. % Recovery | Std. Deviation |
|---|---|---|
| 6519L 1:1 | 0.173 | 0.067 |
| 7017 1:1 | 5.019 | 3.433 |
| 6519L 2:1 | 0.777 | 0.260 |
| 6519L 2:1 10% NMP | 0.397 | 0.251 |

The results of the study provided comparative data for the release profiles of the hydrophilic agent vancomycin hydrochloride. The four (4) screened compositions had varying attributes based on the 1) polymer molecular weight, 2) ratio of diluent, and 3) the usage of a second diluent for further reduced viscosity. Results of the composition preparation indicated that there was not a significant decrease in molecular weight. The resulting 20% and 30% MW decrease for 7017 and 6519L polymers, respectively, over the course of the ˜1 month release study is less than previous studies with the neat polymer and compositions without loaded agent. The rheological properties of these materials performed as expected with an increase in viscosity with a 5% (w/w %) loading of vancomycin hydrochloride and a decreased viscosity with the addition of NMP as shown in FIG. 4A-C.

The primary purpose of the study was the vancomycin release profiles shown in FIG. 5. For the case of 6519L, the following observations were made. The higher diluent (PEG 400) ratio in 6519L 1:1 resulted in steeper initial release profile, particularly over the first 24 hours of in vitro conditions. The release profile of the compositions with the second diluent NMP (6519L 2:1 10% NMP) had similar release profiles to its counterpart without NMP (6519L 2:1), demonstrating comparable release with improved handling/delivery. For 7017, the following observations were made. The decreased initial release rate of the 1:1 ratio as compared to the three other 6519L compositions suggests that the higher molecular weight of the polymeric component may affect the agent release. Extraction analysis indicated the highest percentage of remaining agent of the four compositions.

### Example 9

Purpose: To screen the potential of various PMI technologies to provide release of Erdafitinib (publicly available) extending beyond 1 week. Local therapeutic index and toxicity limits are unknown to PMI, therefore a standard screening approach was taken to determine potential benefit of release technologies. PMI has a range of technologies that may be employed for this purpose, including modified gel and barrier based systems. This is a first screening, and is not an attempt to create a final release system or product.

Scope: Load Erdafitinib into a limited number of proprietary delivery systems and perform a batch release study in artificial urine to determine release and stability of the active agent. Erdafitinib sourced from publicly available supplier having 99% purity. Obtained as fine yellow powder. Viscoprene® used as a hydrosetting gel carrier presenting as a high viscosity liquid. An encapsulant was used to provide a diffusion barrier around the Viscoprene/Erdafitinib matrix for some samples.

Formulation 1: Viscoprene and diluent release matrix. Formulation 2: Viscoprene release matrix. Formulation 3: Viscoprene in Encapsulant A. Formulation 4: Viscoprene in Encapsulant B. Step 1: Prepared release matrices (Erdafitinib in Viscoprene) with or without diluent (active matrix). Each study specimen included approximately 12 mg of drug. Step 2: Active matrix was encapsulated for Formulations 3 and 4. Step 3: Formulations were placed in artificial urine (USP method) at 37° C. Step 4: At time points, artificial urine was changed to fresh artificial urine. Step 5: Samples were analyzed for Erdafitinib content by HPLC.

### Analytical Technique

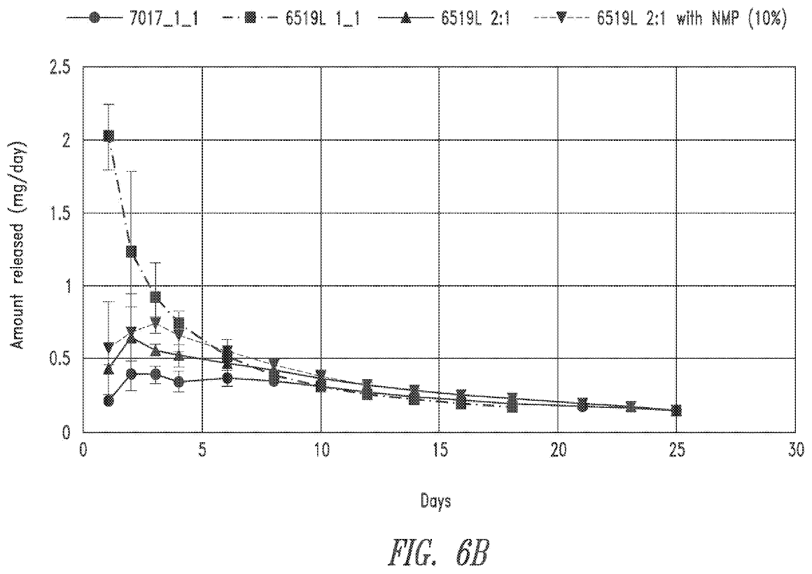

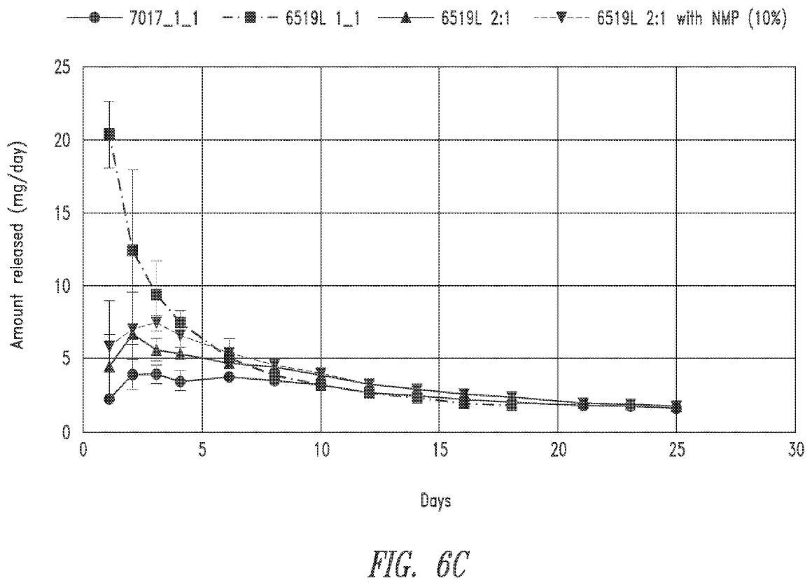

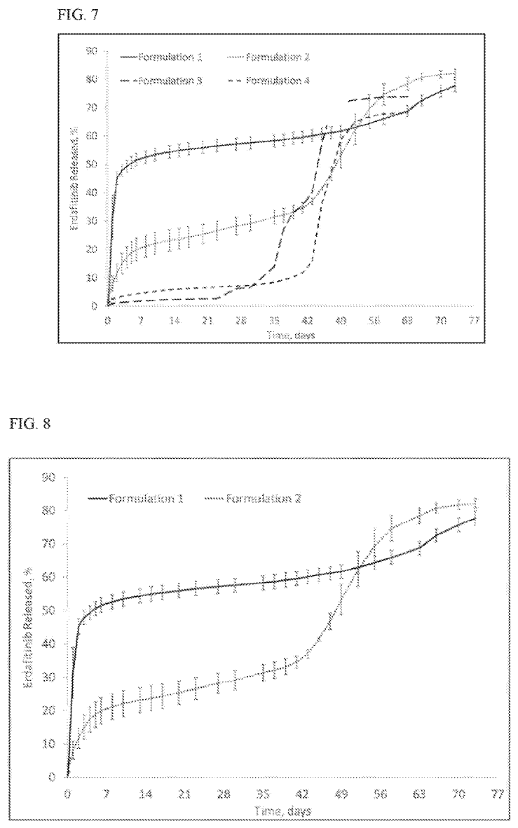

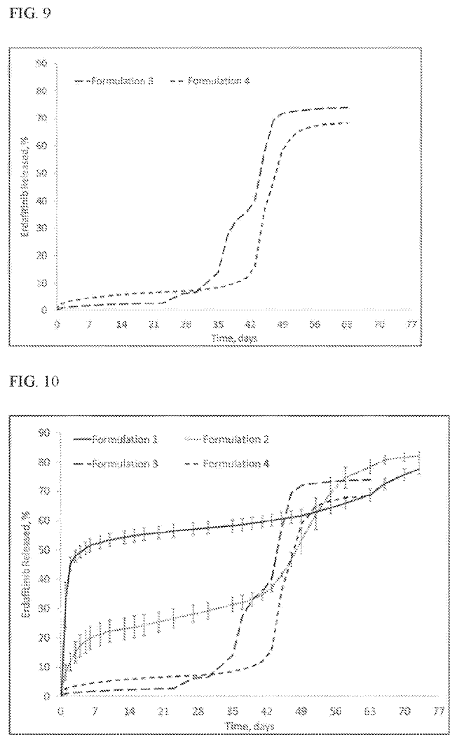

An Isocratic HPLC method (non-stability indicating) able to identify Erdafitinib using a C18 column and water/acetonitrile mobile phase. Calibration curve generated with a R2 value of 0.9999. Erdafitinib in solution provisionally stable over a suitable timeframe (by PDA detection) for in vitro incubation, sampling and analysis. Sample preparation: Erdafitinib mixed readily into Viscoprene matrices. Encapsulation occurred as desired with no noted leakage. Extraction test by HPLC verified accurate loading targets. See FIGS. 7-10. Formulations 1 and 2 are with no encapsulants. Formulations 3 and 4 are encapsulated versions of Formulation 2. Encapsulation is a solid thin-film barrier. Lower 2-day burst release without diluent (12% v/45%). Encapsulants reduced rate of release by 5 to 12 times. Encapsulant in F3 began to fail by day 27. Encapsulant in F4 began to fail by day 43. Late stage release increases likely result of matrix degradation. Comparison of HPLC scans at latest time point match spectral trace of Erdatifinib—material is likely stable through in vitro study for extended duration.

These results represent an exploratory study for a screening of erdafitinib elution from Viscoprene-based extended release systems. Results have not been validated against any standard models and total delivery/daily dosing requirements are not know to the researchers. Erdafitinib can be loaded into a release matrix for extended release. Release matrix can be encapsulated into a more stable form for stability in the study environment. By varying the Viscoprene matrix diluent and encapsulation, the release rate, and in particular the burst release, can be significantly altered. Further system modifications have been identified to increase rate of delivery. Additives may be included in the Viscoprene system to improve linearity of Erdafitinib release rate. Based on study release and anticipated erdafitinib dosage, the system may be capable of delivering a therapeutic dose over an extended duration.

Viscoprene is a good release matrix candidate based on (1) Erdafitinib stability, (2) Erdafitinib solubility, (3) handling performance of matrix-only and encapsulated samples, (4) ability to modulate the release rate using simple system modifications, and (5) potential of the exploratory system to create a form which could be introduced to the bladder (minimally invasively), reside for some length of time during drug delivery, and self-remove or be physician removed. Further exploratory studies, including the preparation of additional system variants and subsequent in vitro release analysis, and a flow controlled simulated bladder model, previously developed by PMI, are contemplated. Study shows the potential of greatly extending the release of Erdafitinib, but one may shorten the release rate and increase linearity of daily dosing. Other aspects include: bladder stability through delivery period; ability to retrieve product; improve release through minimizing burst effect and adjusting daily release target into therapeutic index; ability to deploy product into bladder; low safety risk.

1. An in situ gel-forming composition comprising an absorbable polymer comprising a molecular chain having a ([X—Y—X]—Z)n structure, wherein X represents a relatively hydrophobic polyester block, Y represents a relatively hydrophilic block, Z represents an aliphatic urethane segment and n represents a number of repeating ([X—Y—X]—Z) units, polyethylene glycol, optionally, a second solvent, and at least one bioactive agent, wherein the composition has a viscosity of less than 50,000 cps at room temperature, is biocompatible, and forms a semi-solid mass upon administration to an aqueous environment within a subject in need thereof, and wherein 0.3 percent of total weight of the bioactive agent in the composition to 50 percent of total weight of the bioactive agent in the composition per day is released from the semi-solid mass.

2. The composition of claim 1 wherein the composition comprises two different bioactive agents.

3. The composition of claim 1 wherein the absorbable polymer comprises two different absorbable polymers.

4. The composition of claim 1 wherein the composition comprises two non-identical solvents.

5. (canceled)

6. The composition of claim 1 further comprising an oil selected from corn oil, peanut oil, super refined sesame oil, and super refined peanut oil.

7. (canceled)

8. The composition of claim 1 further comprising tributyl citrate, triethyl citrate, diethyl citrate, acetyl tributyl citrate, or acetyl triethyl citrate.

9. (canceled)

10. The composition of claim 1, wherein the bioactive agent is not disulfram.

11. The composition of claim 1, wherein the bioactive agent is Erdafitinib.

12. The composition of claim 1 wherein the bioactive agent is erdafitinib and the absorbable polymer is 7017, disclosed herein.

13. A method of delivering an effective amount of a bioactive agent to a subject in need thereof comprising administering a composition according to claim 1 to the subject.

14. (canceled)

15. (canceled)

16. (canceled)

17. (canceled)

18. (canceled)

19. A bioactive agent delivery system comprising a delivery vehicle comprising an absorbable polymer, polyethylene glycol, and optionally, a second solvent; and at least one bioactive agent, wherein the delivery system has a viscosity of less than 50,000 cps at room temperature, is biocompatible, and forms a semi-solid mass upon administration to an aqueous environment within a subject in need thereof, and wherein 0.3 percent of total weight of at least one bioactive agent in the composition to 50 percent of total weight of at least one bioactive agent in the composition per day is released from the semi-solid mass.

20. The bioactive agent delivery system of claim 19 wherein the absorbable polymer comprises a poly(ether-ester).

21. The bioactive agent delivery system of claim 19 wherein the absorbable polymer comprises a molecular chain having a X—Y—X or (X—Y)n structure, wherein X represents a relatively hydrophobic polyester block, Y represents a relatively hydrophilic block, and n represents a number of repeating X—Y units.

22. The bioactive agent delivery system of claim 21 wherein the X—Y—X or (X—Y)n is formed by grafting hydrophobic X blocks prepared from monomers selected from the group consisting of glycolide, lactide, ε-caprolactone, p-dioxanone and trimethylene carbonate, to hydrophilic Y blocks selected from the group consisting of polyoxyethylene, poly(oxyethylene-b-oxypropylene), polypeptide polyalkylene oxamate, polysaccharide, derivatives thereof, and liquid, high molecular weight polyether glycols interlinked with an oxalate or succinate functionalities in linear or branched form.

23. The bioactive agent delivery system of claim 19 wherein the absorbable polymer comprises a molecular chain having a ([X—Y—X]—Z)n structure, wherein X represents a relatively hydrophobic polyester block, Y represents a relatively hydrophilic block, Z represents an aliphatic urethane segment and n represents a number of repeating ([X—Y—X]—Z) units,

24. The bioactive agent delivery system of claim 19 wherein the absorbable polymer comprises a segmented aliphatic polyurethane.

25. The bioactive agent delivery system of claim 19 wherein the absorbable polymer comprises a segmented aliphatic polyurethane prepared from lactide and glycolide.

26. The bioactive agent delivery system of claim 19 wherein the absorbable polymer comprises a segmented aliphatic polyurethane comprising polyoxyalkylene glycol chains covalently linked to polyester or polyester-carbonate chain segments, interlinked with aliphatic urethane segments.

27-30. (canceled)
